(19) United States
(12) Patent Application Publication
(10) Pub. No.: US 2010/0286262 A1
(43) Pub. Date: Nov. 11, 2010

(54) PTGS2 HAPLOTYPES

(76) Inventor: Xiao-He Chen, Oxford (GB)

Correspondence Address: MICHAEL BEST & FRIEDRICH LLP, 100 East Wisconsin Avenue, Suite 3300, Milwaukee, WI, 53202, US

(21) Appl. No.: 12/682,356

(22) PCT Filed: Oct. 9, 2008
(86) PCT No.: PCT/GB2008/003433

§ 371 Date: May 13, 2010

(30) Foreign Application Priority Data
Oct. 10, 2007 (GB) 0719775.9

(51) Int. Cl. A61K 31/341; C12Q 1/68; C12N 5/00; C07H 21/00; C40B 40/06; C07D 305/12; A61P 35/00

(52) U.S. Cl. 514/473; 435/6; 435/325; 536/23.1; 536/24.3; 536/24.33; 506/16; 549/323

(57) ABSTRACT

It has been demonstrated that a group of single nucleotide polymorphisms can be used to determine the presence or absence of certain extended PTGS2 haplotypes in the genome of a subject or individual, in order to determine the susceptibility to disease of the subject, the disease prognosis of the subject, or predict the response to therapy of the subject, thereby allowing personalised therapy of said subject.

### CROSS-REFERENCE TO RELATED APPLICATIONS

This patent application is a national stage filing under 35 U.S.C. 371 of International Application No. PCT/GB2008/003433, filed on Oct. 9, 2008, which claims foreign priority benefits to United Kingdom Patent Application No. 0719775.9, filed on Oct. 10, 2007. These applications are incorporated herein by reference in their entireties.

### FIELD OF THE INVENTION

The invention relates to the determining the presence or absence of an extended haplotype in the genome of a subject or individual, in order to determine the susceptibility to disease of the subject, the disease prognosis of the subject, or predict the response to therapy of the subject, thereby allowing personalised therapy of said subject.

### BACKGROUND TO THE INVENTION

Prostaglandin-endoperoxide synthase (PTGS, EC 1.14.99.1), also known as fatty acid cyclooxygenase (COX), is a rate-limiting enzyme in the biosynthesis of prostaglandins and other eicosanoids from arachidonic acid. Previous studies have confirmed the presence of two isoforms of the enzyme, PTGS1 (COX-1, OMIM 176805) and PTGS2 (COX-2, OMIM 600262). Although both isoforms have similar enzymatic activities, PTGS1 is constitutive in the regulation of cell functions, whereas PTGS2 is inducible in response to pro-inflammatory factors, growth factors, peroxisome proliferators and tumour promoters.

The PTGS2 enzyme is encoded by the PTGS2 gene (GenBank accession number NM_{—}000963; SEQ ID NO: 1) which is located on chromosome 1q25.2-25.3, and has 10 exons and 9 introns spanning more than 8.6 kb. Furthermore, over-expression of PTGS2 is thought to promote tumourigenesis via prostaglandin receptor signalling after loss of adenomatous polyposis coli (APC).

### SUMMARY OF THE INVENTION

The present inventors have identified a panel of 18 single nucleotide polymorphism markers (SNPs) that can identify 100% of the haplotypic diversity in the PTGS2 region of a population, based on a sample of 706 colorectal cancer patients. The present inventors have demonstrated the highly informative nature of these SNPs using entropy analysis. The six most informative SNPs from the panel account for 85% of the haplotypic diversity in the PTGS2 region, the ten most informative SNPs account for approximately 97%, and the remaining 6 SNPs account for 3% of the haplotypic diversity in the PTGS2 region. The 18 SNPs are identified as SNP 1 to SNP 18 in Table 3. The sequences of the flanking regions of each SNP are provided as SEQ ID NOS: 2 to 19 respectively as summarised in Table 4.

Based on the panel of 18 SNPs, the inventors have also defined a total of 27 different haplotypes, representing 1318 chromosomes from a sample of 680 subjects. 4 of the 27 haplotypes can be defined by a single SNP from the panel. Collectively these haplotypes represent approximately 45% of the total chromosomes in the subject population. The six most common haplotypes defined by the inventors account for more than 85% of the chromosomes.

The present inventors have demonstrated that genotyping a subject for only 5 SNPs from the panel would be sufficient to identify more than 95% of the available chromosomes without losing statistical power. The five SNPs are SNPs 11, 12, 13, 1 (or 5) and 18 (or 10). Either SNP 1 (rs10911902) or SNP 5 (rs5275) may be used because each of these SNPs can be tagged by the other. Similarly, either SNP 18 (rs6681231) or SNP 10 (rs20417) may be used because each of these SNPs can be tagged by the other.

The present inventors have further identified that detection of extended PTGS2 haplotypes identifiable by the panel of SNPs of the present invention can be used to determine the susceptibility to disease of a subject, to determine the disease prognosis of a subject, or to predict the response to therapy of a subject, thereby allowing personalised therapy of said subject.

Accordingly, the present invention provides a method for determining whether an individual has, or is susceptible to, or will respond to a therapy for a disease selected from a cancer, a cardiovascular disease or a disease that involves the immune system comprising determining whether any of haplotypes 1 to 10 as shown in Table 7 are present in or absent from the genome of the individual.

The present inventors have also demonstrated that genotyping a subject for three SNPs from the panel can identify patients with increased susceptibility to disease, particularly colorectal cancer, and increased positive responses to treatment with a COX-2 inhibitor. The three SNPs are 14 (rs11583191), 16(rs2179555) and 17 (rs10911907). Accordingly, the present invention provides a method for determining whether an individual has, or is susceptible to a cancer, particularly colorectal cancer, and/or will respond positively to COX-2 inhibitor therapy, comprising determining whether the minor alleles of SNPs 14, 16 and 17 are present in or absent from the genome of the individual.

The invention further provides a kit for carrying out the methods of the invention comprising a means for detecting one or more of the polymorphisms that make up any of the haplotypes mentioned herein.

The invention further provides an array for determining the extended PTGS2 haplotype of a subject, comprising a means for detecting one or more of the polymorphisms that make up any of the haplotypes mentioned herein.

### DESCRIPTION OF THE DRAWINGS

FIG. 1 Structure and location of PTGS2 gene on chromosome 1

PTGS2 is located on chromosome 1q25.2-25.3 with 10 exons. Solid blocks in the gene structure represent exons labeled 1 to 10, open blocks untranslated region, curved lines between exons represent introns.

FIG. 2 LD Patterns in the extended PTGS2 region in HapMap CEU panel

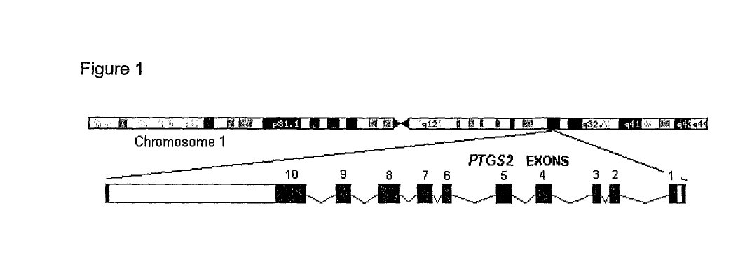

LD structure map shows the strength of pair-wise linkage disequilibrium (LD) in |D′| between SNPs from HapMap reference CEU dataset in A: 500 kb and B: 27 kb regions covering PTGS2. The numbers (1-9) in panel A indicate the LD blocks. The LD block in panel B is part of LD block 6 in panel A. The locations of 11 tagging SNPs are labelled with dbSNP ID accordingly. The number (1-81) is the alternative ID for each of the SNPs in the gene region in sequential order from SNP rs10911902 to SNP rs6681231. The minor allele (MA) frequencies are equal to or more than 5% in CEU. The LD strength is illustrated by shade and number. An empty dark square represents strong LD (|D′|=1.0). Lower LDs are indicated by a square of different shade containing a number. For these squares, said numbers indicate the two digits following the decimal point of the D′ value for the indicated relationship, e.g. 74 corresponds to an LD of 0.74.

FIG. 3 LD Patterns in PTGS2 region in VICTOR patients

LD structure map shows the strength of pair-wise linkage disequilibrium (LD) in |D′| between SNPs from VICTOR dataset in 27 kb region covering PTGS2. The scale bar at the top of the figure shows the location and length of nucleotide sequence in the chromosome, underneath which the location and structure of PTGS2 is illustrated. The SNPs are labeled with dbSNP ID, and their locations are marked accordingly in relation to PTGS2. The number (1-18) is the alternative ID for each of the selected 18 SNPs in VICTOR samples. The LD strength is illustrated by shade and number. An empty dark square represents strong LD (|D′|=1.0). Lower LDs are indicated by a square of different shade containing a number. For these squares, said numbers indicate the two digits following the decimal point of the D′ value for the indicated relationship, e.g. 74 corresponds to an LD of 0.74. Blank space indicates no value for invariant SNP.

FIG. 4 Maximum haplotype diversity explained by the selected SNPs

Percentage of maximum haplotypic diversity is plotted against a subset of SNPs as the size of the subset goes from 1 to 16 SNPs. The individual SNPs are labeled with dbSNP ID on the plot. The percentage is calculated as the entropy of the haplotype frequencies defined by the best n (from 1 to 16) subset of SNPs divided by the maximum entropy of the haplotype frequencies defined by 16 SNPs.

FIG. 5 Overall survival Kaplan-Meier curves for rofecoxib versus placebo

Percentage survival (y axis) is plotted against Years from randomisation (x axis). The hazard ratio (HR) was 0.94, 95% CI: (0.77 to 1.16) P=0.57. There were 177 deaths in the rofecoxib group (solid line) and 191 deaths in the placebo group (dashed line).

FIG. 6 Disease-free survival Kaplan-Meier curves for rofecoxib versus placebo

Percentage disease-free survival (y axis) is plotted against Years from randomisation (x axis). The hazard ratio (HR) was 0.91, 95% CI: (0.78 to 1.07) P=0.25. There were 291 deaths in the rofecoxib group (solid line) and 316 deaths in the placebo group (dashed line). One patient in the placebo group had evidence of disease before randomisation.

FIG. 7 Recurrence analysed by treatment and genotype

Percentage recurrence-free subjects (y axis) is plotted against Years from randomisation (x axis). Dark, solid line indicates variant subjects treated with rofecoxib. Dark, dashed line indicates variant subjects treated with placebo. Light, solid line indicates wild type subjects treated with rofecoxib. Light, dashed line indicates wild type subjects treated with placebo. ‘Wild type’ describes patients having wild type (major) alleles at each of the three SNPs 14, 16 and 17 (rs11583191, rs2179555 and rs10911907). ‘Variant’ describes patients having the variant (minor) allele at all three SNPs.

### DETAILED DESCRIPTION OF THE INVENTION

The present invention relates to examining variation across a whole gene region by selecting a rational panel of known polymorphisms/SNPs which are then genotyped. This allows the comprehensive determination of the genetic architecture of PTGS2 and the identification of particular genetic variants.

The extended PTGS2 haplotype of a subject refers to the combination of polymorphisms located in the extended PTGS2 region of chromosome 1 which are inherited together in the population. The extended PTGS2 region includes the PTGS2 gene itself and its associated up- and down-stream regulatory regions. An extended PTGS2 haplotype can be defined by sets of single nucleotide polymorphism markers (SNPs) that are inherited together in blocks.

The present invention relates to a method of determining disease susceptibility, disease prognosis, or predicted response to therapy due to the absence or presence of a particular extended PTGS2 haplotype in a subject.

The subject is typically a human from a Caucasian population, a Chinese population or an African population. The subject may be suspected of being at risk of disease, may have already been diagnosed as having a disease, may be awaiting treatment for a disease, or may have been treated for a disease which has subsequently gone into remission.

The subject may be suspected of being at risk of disease because of exposure to known genetic or environmental risk factors, or because of the opinion of medical practitioners.

The disease is typically a disease which is responsive to treatment with non-steroidal anti-inflammatory drug (NSAID) treatment, particularly PTGS2-specific NSAID treatment. The disease is typically a cancer, osteoarthritis, Alzheimers disease, an infectious disease, a cardiovascular disease, an inflammatory disease, a musculoskeletal disease or a disease that involves the immune system. The cardiovascular disease may be a myocardial infarction or other cardiovascular disease. The cancer may be colorectal cancer, breast cancer, prostate cancer, esophageal cancer, stomach cancer, liver cancer or lung cancer. The cancer is typically colorectal cancer.

After colorectal cancer has been diagnosed, tests are done to determine the stage of the disease. It is important to know the stage of the disease in order to plan the best treatment. The following stages are used for colon cancer:

Stage 0 (Carcinoma in Situ): In stage 0, the cancer is found in the innermost lining of the colon only. Stage 0 cancer is also called carcinoma in situ.

Stage I: In stage I, the cancer has spread beyond the innermost lining of the colon to the second and third layers and involves the inside wall of the colon, but it has not spread to the outer wall of the colon or outside the colon. Stage I colon cancer is sometimes called Dukes' A colon cancer.

Stage II: In stage II, cancer has spread outside the colon to nearby tissue, but it has not gone into the lymph nodes. (Lymph nodes are small, bean-shaped structures that are found throughout the body. They filter substances in a fluid called lymph and help fight infection and disease.) Stage II colon cancer is sometimes called Dukes' B colon cancer.

Stage III: In stage III, cancer has spread to nearby lymph nodes, but it has not spread to other parts of the body. Stage III colon cancer is sometimes called Dukes' C colon cancer.

Stage IV: In stage IV, cancer has spread to other parts of the body, such as the liver or lungs. Stage IV colon cancer is sometimes called Dukes' D colon cancer.

The present invention provides a method for identifying the prognosis of a subject who has colorectal cancer based on their extended PTGS2 haplotype.

The present invention also provides a method for predicting the response of a subject to COX-2 inhibitor treatment.

### Selection of Polymorphisms

Informative SNPs in a region, referred to as haplotype tagging SNPs, can be identified by mathematical analysis of databases of genotype information. For example, the software program Haploview implements the Tagger pairwise-tagging algorithm, which can directly communicate with HapMap reference dataset (http://www.hapmap.org/), and is available both as an online programme and as stand-alone software. The HapMap reference dataset includes SNP genotypes of 90 individuals from 30 parent-parent-offspring trios of European descent from Utah (CEU). Potential haplotype tagging SNPs can also be identified by analysis of literature for findings of an association between a SNP and a particular phenotype, for example increased disease susceptibility. For example, six putatively functional SNPs in the extended PTGS2 region are rs689469, rs4648298, rs5275, rs20432, rs5277 and rs20417.

### Detection of Polymorphisms

The detection or genotyping of polymorphisms according to the invention is may comprise contacting a polynucleotide of the subject with a specific binding agent for a polymorphism and determining whether the agent binds to the polynucleotide, wherein binding of the agent indicates the presence of the polymorphism, and lack of binding of the agent indicates the absence of the polymorphism.

The method is generally carried out in vitro on a sample from the subject. The sample typically comprises a body fluid and/or cells of the individual and may, for example, be obtained using a swab, such as a mouth swab. The sample may be a blood, urine, saliva, skin, cheek cell or hair root sample. The sample is typically processed before the method is carried out, for example DNA extraction may be carried out. The polynucleotide or protein in the sample may be cleaved either physically or chemically, for example using a suitable enzyme. In one embodiment the part of polynucleotide in the sample is copied or amplified, for example by cloning or using a PCR based method prior to detecting the polymorphism.

In the present invention, any one or more methods may comprise determining the presence or absence of one or more polymorphisms in the subject. The polymorphism is typically detected by directly determining the presence of the polymorphic sequence in a polynucleotide or protein of the subject. Such a polynucleotide is typically genomic DNA, mRNA or cDNA. The polymorphism may be detected by any suitable method such as those mentioned below.

A specific binding agent is an agent that binds with preferential or high affinity to the polynucleotide having the polymorphism but does not bind or binds with only low affinity to other polynucleotides. The specific binding agent may be a probe or primer. The probe may be a protein (such as an antibody) or an oligonucleotide. The probe may be labelled or may be capable of being labelled indirectly. The binding of the probe to the polynucleotide or protein may be used to immobilise either the probe or the polynucleotide or protein.

Generally in the method, determination of the binding of the agent to the polymorphism can be carried out by determining the binding of the agent to the polynucleotide of the subject. However in one embodiment the agent is also able to bind the corresponding wild-type sequence, for example by binding the nucleotides which flank the polymorphism position, although the manner of binding to the wild-type sequence will be detectably different to the binding of a polynucleotide containing the polymorphism.

The method may be based on an oligonucleotide ligation assay in which two oligonucleotide probes are used. These probes bind to adjacent areas on the polynucleotide which contains the polymorphism, allowing after binding the two probes to be ligated together by an appropriate ligase enzyme. However the presence of single mismatch within one of the probes may disrupt binding and ligation. Thus ligated probes will only occur with a polynucleotide that contains the polymorphism, and therefore the detection of the ligated product may be used to determine the presence of the polymorphism.

In one embodiment the probe is used in a heteroduplex analysis based system. In such a system when the probe is bound to polynucleotide sequence containing the polymorphism it forms a heteroduplex at the site where the polymorphism occurs and hence does not form a double strand structure. Such a heteroduplex structure can be detected by the use of single or double strand specific enzyme. Typically the probe is an RNA probe, the heteroduplex region is cleaved using RNAase H and the polymorphism is detected by detecting the cleavage products.

The method may be based on fluorescent chemical cleavage mismatch analysis which is described for example in PCR Methods and Applications 3, 268-71 (1994) and Proc. Natl. Acad. Sci. 85, 4397-4401 (1998).

In one embodiment a PCR primer is used that primes a PCR reaction only if it binds a polynucleotide containing the polymorphism, for example a sequence- or allele-specific PCR system, and the presence of the polymorphism may be determined by the detecting the PCR product. Preferably the region of the primer which is complementary to the polymorphism is at or near the 3′ end of the primer. The presence of the polymorphism may be determined using a fluorescent dye and quenching agent-based PCR assay such as the Taqman PCR detection system. The specific binding agent may be capable of specifically binding the amino acid sequence encoded by a variant sequence. For example, the agent may be an antibody or antibody fragment. The detection method may be based on an ELISA system. The method may be an RFLP based system. This can be used if the presence of the polymorphism in the polynucleotide creates or destroys a restriction site that is recognised by a restriction enzyme.

The presence of the polymorphism may be determined based on the change which the presence of the polymorphism makes to the mobility of the polynucleotide or protein during gel electrophoresis. In the case of a polynucleotide single-stranded conformation polymorphism (SSCP) or denaturing gradient gel electrophoresis (DDGE) analysis may be used.

The presence of the polymorphism may be detected by means of fluorescence resonance energy transfer (FRET). In particular, the polymorphism may be detected by means of a dual hybridisation probe system. This method involves the use of two oligonucleotide probes that are located close to each other and that are complementary to an internal segment of a target polynucleotide of interest, where each of the two probes is labelled with a fluorophore. Any suitable fluorescent label or dye may be used as the fluorophore, such that the emission wavelength of the fluorophore on one probe (the donor) overlaps the excitation wavelength of the fluorophore on the second probe (the acceptor). A typical donor fluorophore is fluorescein (FAM), and typical acceptor fluorophores include Texas red, rhodamine, LC-640, LC-705 and cyanine 5 (Cy5).

In order for fluorescence resonance energy transfer to take place, the two fluorophores need to come into close proximity on hybridisation of both probes to the target. When the donor fluorophore is excited with an appropriate wavelength of light, the emission spectrum energy is transferred to the fluorophore on the acceptor probe resulting in its fluorescence. Therefore, detection of this wavelength of light, during excitation at the wavelength appropriate for the donor fluorophore, indicates hybridisation and close association of the fluorophores on the two probes. Each probe may be labelled with a fluorophore at one end such that the probe located upstream (5′) is labelled at its 3′ end, and the probe located downstream (3′) is labelled at is 5′ end. The gap between the two probes when bound to the target sequence may be from 1 to 20 nucleotides, preferably from 1 to 17 nucleotides, more preferably from 1 to 10 nucleotides, such as a gap of 1, 2, 4, 6, 8 or 10 nucleotides. The first of the two probes may be designed to bind to a conserved sequence of the gene adjacent to a polymorphism and the second probe may be designed to bind to a region including one or more polymorphisms. Polymorphisms within the sequence of the gene targeted by the second probe can be detected by measuring the change in melting temperature caused by the resulting base mismatches. The extent of the change in the melting temperature will be dependent on the number and base types involved in the nucleotide polymorphisms.

The polymorphic position may be typed directly, in other words by determining the nucleotide present at that position, or indirectly, for example by determining the nucleotide present at another polymorphic position that is in linkage disequilibrium with said polymorphic position. Polymorphisms which are in linkage disequilibrium with each other in a population are typically found together on the same chromosome. Typically one is found at least 30% of the times, for example at least 40%, at least 50%, at least 70% or at least 90%, of the time the other is found on a particular chromosome in individuals in the population. Thus a polymorphism which is not a functional susceptibility polymorphism, but is in linkage disequilibrium with a functional polymorphism, may act as a marker indicating the presence of the functional polymorphism.

Polymorphisms which are in linkage disequilibrium with the polymorphisms mentioned herein are typically located within 500 kb, preferably within 400 kb, within 200 kb, within 100 kb, within 50 kb, within 10 kb, within 5 kb, within 1 kb, within 500 bp, within 100 bp, within 50 bp or within 10 bp of the polymorphism.

A polynucleotide of the invention may be used as a primer, for example for PCR, or a probe. A polynucleotide or polypeptide of the invention may carry a revealing label. Suitable labels include radioisotopes such as 32P or 35S, fluorescent labels, enzyme labels or other protein labels such as biotin.

Polynucleotides of the invention may be used as a probe or primer which is capable of selectively binding to a polymorphism. The invention thus provides a probe or primer for use in a method according to the invention, which probe or primer is capable of selectively detecting the presence of a polymorphism. Preferably the probe is isolated or recombinant nucleic acid. The probe may be immobilised on an array, such as a polynucleotide array.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of a full length polynucleotide sequence of the invention.

The polynucleotides (e.g. primer and probes) of the invention may be present in an isolated or substantially purified form. They may be mixed with carriers or diluents which will not interfere with their intended use and still be regarded as substantially isolated. They may also be in a substantially purified form, in which case they will generally comprise at least 90%, e.g. at least 95%, 98% or 99%, of the polynucleotides or dry mass of the preparation.

In the method of the invention the presence or absence of the alleles mentioned in Table 7 may be detected by any suitable means. Typically in the method one or more of the polymorphisms listed in Table 7 is typed. Thus, the presence or absence of the polymorphism may be determined, typically in a polynucleotide from the subject, to ascertain whether or not the genome of the subject comprises the relevant polymorphism. In one embodiment, whether or not the genome of the subject comprises all of the polymorphisms listed a row of Table 7 is ascertained.

In a preferred embodiment, at least 1, 2, 3, 4, 5, 6, 7, 8, 9, 10, 11, 12, 13, 14, 15, 16, 17 or 18 of the polymorphisms shown in Table 7 are typed in the method of the invention. In one embodiment, a polymorphism which is in linkage to disequilibrium with a polymorphism shown in Table 7 is typed (in order to acesertain the presence of a polymorphism in Table 7 in the genome of the subject). In one embodiment, whether or not the polymorphisms which are typed are present on the same DNA strand is also determined.

In an alternative embodiment, polymorphisms are detected by sequencing the extended PTGS2 region, or a part thereof, using a massively-parallel system. For example a sequencing-by-synthesis approach. In a preferred embodiment, the part of the PTGS2 region to be sequenced includes the PTGS2 gene and its regulatory regions.

In another embodiment, at least SNPs 14, 16 and 17 (rs11583191, rs2179555 and rs10911907) are typed in the method of the invention. In this embodiment, a subject having the major allele in all three SNPs is identified as having a better disease prognosis than a patient having the minor allele in all three SNPs. The disease is typically cancer, most typically colorectal cancer.

In a further embodiment, at least SNPs 14, 16 and 17 (rs11583191, rs2179555 and rs10911907) are typed in the method of the invention. The subject being typed may be suspected of being at risk of disease, may have already been diagnosed as having a disease, may be awaiting treatment for a disease, or may have been treated for a disease which has subsequently gone into remission. The subject may be suspected of being at risk or may have been diagnosed as having a disease according to another embodiment of the present invention, or by any other suitable means. The disease is typically cancer, most typically colorectal cancer. In this embodiment, a subject having the major allele in all three SNPs is identified as being less likely to respond positively to COX-2 inhibitor treatment disease prognosis than a patient having the minor allele in all three SNPs. The COX-2 inhibitor is typically a COX-2 specific inhibitor. By COX-2 specific, it will be understood that this means the inhibitor is at least 5 fold more potent at inhibiting COX-2 than COX-1. Preferred COX-2 inhibitors may have the general formula I:

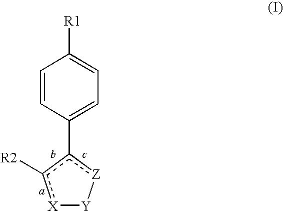

or pharmaceutically acceptable salts thereof wherein:

X—Y—Z— is selected from the group consisting of:
(a) —CH2CH2CH2-,
(b) —C(O)CH2CH2-,
(c) —CH2CH2C(O)—,
(d) —CR5(R5′)—O—C(O)—,
(e) —C(O)—O—CR5(R5′)—,
(f) —CH2-NR3-CH2-,
(g) —CR5(R5′)—NR3-C(O)—,
(h) —CR4=CR4′—S—,
(i) —S—CR4=CR4′—,
(j) —S—N═CH—,
(k) —CH═N—S—,
(l) —N═CR4-O—,
(m) —O—CR4=N—
(n) —N═CR4-NH—;
(o) —N═CR4-S—, and
(p) —S—CR4=N—;
(q) —C(O)—NR3-CR5(R5′)—;
(r) —R3N—CH═CH— provided R1 is not —S(O)2Me
(s) —CH═CH—NR3- provided R1 is not —S(O)2Me

when side b is a double bond, and sides a an c are single bonds; and

X—Y—Z— is selected from the group consisting of:
(a) ═CH—O—CH═, and
(b) ═CH—NR3-CH═,
(c) ═N—S—CH═,
(d) ═CH—S—N═,
(e) ═N—O—CH═,
(f) ═CH—O—N═,
(g) ═N—S—N═,
(h) ═N—O—N═,

when sides a and c are double bonds and side b is a single bond;

R1 is selected from the group consisting of
(a) S(O)2CH3,
(b) S(O)2NH2,
(c) S(O)2NHC(O)CF3,
(d) S(O)(NH)CH3,
(e) S(O)(NH)NH2,
(f) S(O)(NH)NHC(O)CF3,
(g) P(O)(CH3)OH, and
(h) P(O)(CH3)NH2,

R2 is selected from the group consisting of
(a) C1-6alkyl,
(b) C3, C4, C5, C6, and C7, cycloalkyl,
(c) mono-, di- or tri-substituted phenyl or naphthyl wherein the substituent is selected from the group consisting of

(1) hydrogen,

(2) halo,

(3) C1-6alkoxy,

(4) C1-6alkylthio,

(5) CN,

(6) CF3,

(7) C1-6alkyl,

(8) N3,

(9) —CO2H,

(10) —CO2-C1-4alkyl,

(11) —C(R5)(R6)-OH,

(12) —C(R5)(R6)-O—C1-4alkyl, and

(13) —C1-6alkyl-CO2-R5;
(d) mono-, di- or tri-substituted heteroaryl wherein the heteroaryl is a monocyclic aromatic ring of 5 atoms, said ring having one hetero atom which is S, O, or N, and optionally 1, 2, or 3 additionally N atoms; or the heteroaryl is a monocyclic ring of 6 atoms, said ring having one hetero atom which is N, and optionally 1, 2, 3, or 4 additional N atoms; said substituents are selected from the group consisting of

(1) hydrogen,

(2) halo, including fluoro, chloro, bromo and iodo,

(3) C1-6alkyl,

(4) C1-6alkoxy,

(5) C1-6alkylthio,

(6) CN,

(7) CF3,

(8) N3,

(9) —C(R5)(R6)-OH, and

(10) —C(R5)(R6)-O—C1-4alkyl;
(e) benzoheteroaryl which includes the benzo fused analogs of (d);

R3 is selected from the group consisting of
(a) hydrogen,
(b) CF3,
(c) CN,
(d) C1-6alkyl,
(e) hydroxyC1-6alkyl,
(f) —C(O)—C1-6alkyl,
(g) optionally substituted

(1) —C1-5 alkyl-Q,

(2) —C1-3 alkyl-O—C1-3 alkyl-Q,

(3) —C1-3 alkyl-S—C1-3 alkyl-Q,

(4) —C1-5 alkyl-O-Q, or

(5) —C1-5 alkyl-S-Q,

wherein the substituent resides on the alkyl and the substituent is C1-3alkyl;
(h) -Q

R4 and R4′ are each independently selected from the group consisting of
(a) hydrogen,
(b) CF3,
(c) CN,
(d) C1-6alkyl,
(e) -Q,
(f) —O-Q;
(g) —S-Q, and
(h) optionally substituted

(1) —C1-5 alkyl-Q,

(2) —O—C1-5 alkyl-Q,

(3) —S—C1-5 alkyl-Q,

(4) —C1-3 alkyl-O—C1-3 alkyl-Q,

(5) —C1-3 alkyl-S—C1-3 alkyl-Q,

(6) —C1-5 alkyl-O-Q,

(7) —C1-5 alkyl-S-Q,

wherein the substituent resides on the alkyl and the substituent is C1-3alkyl, and

R5, R5′, R6, R7 and R8 are each independently selected from the group consisting of
(a) hydrogen,
(b) C1-6alkyl,
or R5 and R6 or R7 and R8 together with the carbon to which they are attached form a saturated monocyclic carbon ring of 3, 4, 5, 6 or 7 atoms;

Q is CO2H, CO2-C1-4alkyl, tetrazolyl-5-yl, C(R7)(R8)(OH), or

C(R7)(R8)(O—C1-4alkyl);

provided that when X—Y—Z is —S—CR4=CR4′, then R4 and R4′ are other than CF3.

Particularly preferred COX-2 inhibitors include rofecoxib, that is 3-phenyl-4-(4(methylsulfonyl)phenyl)-2-(5H)-furanone. Other examples of suitable COX-2 inhibitors include valdecoxib and celecoxib.

### Detection Kit

The invention also provides a kit that comprises means for determining the presence or absence of one or more polymorphisms in a subject which define the extended PTGS2 haplotype or disease susceptibility of the subject. In particular, such means may include a specific binding agent, probe, primer, pair or combination of primers, or antibody, including an antibody fragment, as defined herein which is capable of detecting or aiding detection of a polymorphism. The primer or pair or combination of primers may be sequence specific primers which only cause PCR amplification of a polynucleotide sequence comprising the polymorphism to be detected, as discussed herein. The kit may also comprise a specific binding agent, probe, primer, pair or combination of primers, or antibody which is capable of detecting the absence of the polymorphism. The kit may further comprise buffers or aqueous solutions.

The kit may additionally comprise one or more other reagents or instruments which enable any of the embodiments of the method mentioned above to be carried out. Such reagents or instruments may include one or more of the following: a means to detect the binding of the agent to the polymorphism, a detectable label such as a fluorescent label, an enzyme able to act on a polynucleotide, typically a polymerase, restriction enzyme, ligase, RNAse H or an enzyme which can attach a label to a polynucleotide, suitable buffer(s) or aqueous solutions for enzyme reagents, PCR primers which bind to regions flanking the polymorphism as discussed herein, a positive and/or negative control, a gel electrophoresis apparatus, a means to isolate DNA from sample, a means to obtain a sample from the individual, such as swab or an instrument comprising a needle, or a support comprising wells on which detection reactions can be carried out. The kit may be, or include, an array such as a polynucleotide array comprising the specific binding agent, preferably a probe, of the invention. The kit typically includes a set of instructions for using the kit.

### Detection Array

The invention also provides an array that comprises means for determining the presence or absence of one or more polymorphisms in a subject which define the extended PTGS2 haplotype of the subject.

The array typically comprises probes or primers which are capable of selectively binding to the polymorphisms of the invention. The probes or primers are immobilised on a solid-surface such as a glass slide. Such primers or probes will preferably be at least 10, preferably at least 15 or at least 20, for example at least 25, at least 30 or at least 40 nucleotides in length. Typically an array will consist of 100, 1000, 10000, 100000, 1×10⁶ or 1×10⁷ different probes or primers.

Detection of the presence or absence of one or more polymorphisms in a subject using the array may be performed by any appropriate method. Typically such a method comprises taking a sample of genomic DNA from a subject, fragmenting the sample using, e.g. a restriction enzyme, and labelling the resulting fragments with a detectable marker such as a fluorescent label, which enables detection of the fragment when bound to the probes or primers of the array. The labelled fragments are incubated with the array under conditions suitable for hybridisation between the immobilised probes and the labelled fragments to occur. The genomic DNA sample may be amplified by any suitable non-specific amplification method prior to fragmenting.

### Screening for Therapeutic Agents

The present invention also relates to a method of screening for a therapeutic substance which treats a disease as mentioned herein. In one embodiment, the method comprising contacting a candidate substance with a vector that comprises the sequence of any of the haplotypes of the invention and determining whether the candidate substance is able to modulate expression from the vector. In a preferred embodiment, the vector comprises the sequence of any of haplotypes 1 to 10.

The vector for use in the present invention group consists of bacterial vectors, virus vectors, nucleic acid based vectors and the like. The virus vectors include but are not limited to poxvirus, adenovirus, herpes virus, alphavirus, retrovirus, picornavirus, iridovirus and the like. The vector may express in a cell-free system, for example using commercially available in vitro transcription and translation systems. Alternatively the vector may be transfected into a non-human cell for expression. Transfection may be performed by any standard method, for example, electroporation, heat shock, or treatment with a suitable transfection reagent.

The vector may additionally comprise a marker to confirm expression. Suitable markers include antibiotic resistance genes, genes encoding fluorescent proteins or genes encoding reporter enzymes.

Modulation of expression from the vector by the candidate substance may be quantified by comparison to modulation of expression achieved by administration of a NSAID to the vector. The NSAID may be PTGS2-specific.

In a further embodiment the invention provides a method of screening as above, wherein the candidate substance is able to modulate expression of the polypeptide expressed by a gene comprised in a particular haplotype in the vector. Any suitable assay format can be used to determine whether the candidate agent modulates the expression or activity of the polypeptide. The method may be carried out in vitro or in vivo. In one embodiment the method is carried out on a non-human cell, cell culture or cell extract that comprises the polypeptide.

In another embodiment, the method comprises contacting a candidate substance with a polypeptide expressed by a gene comprised in a particular haplotype and determining whether the candidate substance is able to modulate the activity of the polypeptide. Any suitable assay format can be used to determine whether the candidate agent modulates the activity of the polypeptide. The method may be carried out in vitro or in vivo. In one embodiment the method is carried out on a non-human cell, cell culture or cell extract that comprises the polypeptide.

The method may also be carried out in vivo in a non-human subject which is transgenic for a haplotype as defined herein. The transgenic non-human subject is typically of a species commonly used in biomedical research and is preferably a laboratory strain. Suitable subjects include rodents, particularly a mouse, rat, guinea pig, ferret, gerbil or hamster. Most preferably the subject is a mouse.

Suitable candidate agents which may be tested in the above screening methods include antibody agents, for example monoclonal and polyclonal antibodies, single chain antibodies, chimeric antibodies and CDR-grafted antibodies. Furthermore, combinatorial libraries, defined chemical identities, peptide and peptide mimetics, oligonucleotides and natural agent libraries, such as display libraries may also be tested. The candidate agents may be chemical compounds, which are typically derived from synthesis around small molecules which may have any of the properties of the agent mentioned herein. Batches of the candidate agents may be used in an initial screen of, for example, ten substances per reaction, and the substances of batches which show modulation tested individually. The term ‘agent’ is intended to include a single substance and a combination of two, three or more substances. For example, the term agent may refer to a single peptide, a mixture of two or more peptides or a mixture of a peptide and a defined chemical entity.

The invention is illustrated by the following Examples:

### Example 1

It is generally accepted that genetic variation within the PTGS2 gene may moderate disease susceptibility. However, although some research has been carried out on functional characterisation of PTGS2 polymorphisms, the effects of each known genetic variant on enzyme function have not yet been fully characterised. This renders it difficult to predict a priori which allelic variants may be associated with cancer risk or survival. To what extent the inherited variability in the PTGS2 gene is predictive of individual variation in elevated levels of PTGS2 expression in tumour cells also remains to be determined. Previous studies have demonstrated that genetic variation in the PTGS2 gene was associated with both reduced, or increased risk of developing cancer, depending on the specific variant and cancer type under study. The majority of published studies on PTGS2 polymorphisms are mainly common variant, cancer susceptibility studies, with limited overlap of the specific SNPs under study.

Which particular genetic variants might be of importance as prognostic or predictive factors of cancer survival is also unknown at this point. There has been no unified analysis of genetic variation across the extended gene region, including the regulatory regions. This could be achieved by large scale sequencing to identify existing and novel genetic variants but even for a small gene (e.g. less than 10 kb), genotyping and/or re-sequencing all possible variants in the gene and flanking region in a large population is both laborious and costly. The present invention, as exemplified below, does not suffer from these limitations.

The extended PTGS2 region covers approximately 500 kb. The inventors have demonstrated that this region lies between two adjacent recombination hotspots and has little evidence for historical recombination. In general, the region is therefore a homogeneous block in strong LD. For comparison, the inventors also analysed the region in the Chinese population (CHB) and African population (YRI). A similar pattern was also seen in the Chinese population (CHB), although a more heterogeneous block in the African population (YRI) has been found.

As exemplified below, the present invention demonstrates that the less complex genetic structure in the extended PTGS2 region allows a small number of SNPs or even a single SNP to be used to mark an expanded stretch of haplotype, particularly in the Caucasian and Chinese populations. For instance, a minimum of 7 tagging SNPs (with their minor allele frequencies above 5% at r²≧0.8) are needed to capture most of the haplotypic information in PTGS2 region (spanning ˜30 kb) in the Caucasian population (CEU), while only 4 tagging SNPs were needed in the Chinese population (CHB). Even for the more heterogeneous African population, only 16 tagging SNPs need to be genotyped to capture a similar amount of information. The inventors have identified one SNP (rs5275) as being particularly informative. This marker alone can capture 27% of the diversity. Combining SNPs rs12042763 and rs689466 with rs5275, it could capture 47% and 60% of the diversity respectively.

From a total of 27 different extended PTGS2 haplotypes identified by the inventors as being present in the Caucasian population, 4 out of 27 haplotypes can be denoted by a single SNP, and represent ˜45% of the total chromosomes in the patient population. For example, the most common haplotype is denoted by a single tagging SNP rs689466, and represents ˜20% of the total chromosomes. The 6 most common haplotypes (>10%) account for more than 85% of the chromosomes.

The present inventors have shown that a small panel of informative SNPs can capture most of the haplotypic diversity in a haplotype block of reasonable size or length such as the extended PTGS2 region. The date presented herein indicates that genotyping 5 tagging SNPs in the extended PTGS2 gene region is enough to cover more than 95% of chromosomes, without loss of power. These five tagging SNPs are preferably rs689466, rs12042763, rs2725559, rs10911902 (or rs5275) and rs6681231 (rs20417).

Either rs10911902 or rs5275 may be used because each of these SNPs can be tagged by the other. Similarly, either rs6681231 or rs20417 may be used because each of these SNPs can be tagged by the other.

### Materials and Methods

Unless otherwise indicated, statistical p values are generated by Pearson's chi square test. In all cases, a specific SNP, haplotype or group of haplotypes is compared against all other SNPs, haplotypes or groups of haplotypes as appropriate.

### Subjects and SNP Genotype Datasets

The reference dataset of SNP genotypes were chosen from HapMap European CEU panels (release #21/Phase II July 2006, http://www.hapmap.org/). This dataset includes SNP genotypes of 90 individuals from 30 parent-parent-offspring trios of European descent from Utah (CEU). For the purpose of comparison, we also analysed HapMap Chinese population (CHB, n=45) and African population (YRI, n=90).

The testing dataset of SNP genotypes were from 706 colorectal cancer patients in the VICTOR Trial, which is a phase III, randomised, double-blind, placebo-controlled trial of Vioxx® (rofecoxib) in colorectal cancer patients following potentially curative therapy (http://www.octo-oxford.org.uk/). All subjects gave informed written consent and the project was approved by the local research ethics committee. Primers were designed and genotyping was performed according to standard protocols using Amplifluor™ fluorescent allele specific genotyping methods. The specific primers used in these experiments are set out in Table 1:

TABLE 1

| SNP | | Primer | Primer | Primer | | |
|---|---|---|---|---|---|---|
| ID | dbSNP ID | (allele 1) | (allele 2) | (common) | Allele 1 | Allele 2 |
| 1 | rs10911902 | GAAGGTGA | GAAGGTC | TCTTAATG | T | C |
| | | CCAAGTTC | GGAGTCAA | GGTGGTC | | |
| | | ATGCTAGT | CGGATTGT | CTATGTTT | | |
| | | CCTAGAAT | CCTAGAAT | GTGAT | | |
| | | CTGCACTA | CTGCACTA | | | |
| | | ATCACTA | ATCACTG | | | |
| 2 | rs689469 | GAAGGTGA | GAAGGTC | CTTTTGGG | A | G |
| | | CCAAGTTC | GGAGTCAA | AAGAGGG | | |
| | | ATGCTACT | CGGATTAC | AGAAAATG | | |
| | | ATTTCTTT | TATTTCTTT | AAATAAA | | |
| | | CTTAACCT | CTTAACCT | | | |
| | | TAAACATT | TAAACATT | | | |
| | | CTAAAT | CTAAAC | | | |
| 3 | rs4648298 | GAAGGTGA | GAAGGTC | TTATGTCT | A | G |
| | | CCAAGTTC | GGAGTCAA | TATTAGGA | | |
| | | ATGCTAAA | CGGATTAT | CACTATGG | | |
| | | ATCAATGA | CAATGATT | TTATAA | | |
| | | TTGTAGGC | GTAGGCTT | | | |
| | | TTAAACAC | AAACACAGC | | | |
| | | AGT | | | | |
| 4 | rs2206593 | GAAGGTGA | GAAGGTC | GATTTTGC | A | G |
| | | CCAAGTTC | GGAGTCAA | TATGAGGT | | |
| | | ATGCTGTA | CGGATTAC | TAATGAAG | | |
| | | CAACAGAA | AACAGAAA | TACCAA | | |
| | | AATCTGAG | ATCTGAGA | | | |
| | | AAAACATA | AAACATAT | | | |
| | | TCA | CG | | | |
| 5 | rs5275 | GAAGGTGA | GAAGGTC | TTAATAAT | T | C |
| | | CCAAGTTC | GGAGTCAA | GCACTGAT | | |
| | | ATGCTACT | CGGATTAC | ACCTGYTT | | |
| | | AATGTTTG | TAATGTTT | TTGTTT | | |
| | | AAATTTTA | GAAATTTT | | | |
| | | AAGTACTT | AAAGTACT | | | |
| | | TTGGTT | TTTGGTC | | | |
| 6 | rs5272 | GAAGGTGA | GAAGGTC | GCTTTTCT | A | G |
| | | CCAAGTTC | GGAGTCAA | ACCAGAAG | | |
| | | ATGCTATG | CGGATTAT | GGCAGGA | | |
| | | GTGACATC | GGTGACAT | TA | | |
| | | GATGCTGT | CGATGCTG | | | |
| | | GGA | TGGG | | | |
| 7 | rs20432 | GAAGGTGA | GAAGGTC | TGGATTTC | G | T |
| | | CCAAGTTC | GGAGTCAA | AATAGCAT | | |
| | | ATGCTATT | CGGATTAT | AGCTTCAA | | |
| | | ATATACTT | ATACTTCA | GTTATT | | |
| | | CACTATGA | CTATGATG | | | |
| | | TGATATGG | ATATGGTA | | | |
| | | TAATTC | ATTA | | | |
| 8 | rs5277 | GAAGGTGA | GAAGGTC | CCCTTCCT | G | C |
| | | CCAAGTTC | GGAGTCAA | TCGAAATG | | |
| | | ATGCTGAA | CGGATTGA | CAATTATG | | |
| | | AGACACTT | AAGACACT | AGTTA | | |
| | | GTACTTAC | TGTACTTA | | | |
| | | ATGTCAAC | CATGTCAAG | | | |
| 9 | rs4648261 | GAAGGTGA | GAAGGTC | TTGCTTAT | A | G |
| | | CCAAGTTC | GGAGTCAA | CTTAAAAC | | |
| | | ATGCTATT | CGGATTGA | ATAATCCG | | |
| | | GAGTGGAA | GTGGAAGA | GGCTTT | | |
| | | GAAGTTGT | AGTTGTTT | | | |
| | | TTTAAATAT | TAAATATT | | | |
| | | TCTAA | CTAG | | | |
| 10 | rs20417 | GAAGGTGA | GAAGGTC | GGACCAGT | G | C |
| | | CCAAGTTC | GGAGTCAA | ATTATGAG | | |
| | | ATGCTCCT | CGGATTCC | GAGAATTT | | |
| | | TGTTTCTT | TTGTTTCT | ACCTT | | |
| | | GGAAAGA | TGGAAAGA | | | |
| | | GAGGC | GAGGG | | | |
| 11 | rs689466 | GAAGGTGA | GAAGGTC | GAGCACTA | A | G |
| | | CCAAGTTC | GGAGTCAA | CCCATGAT | | |
| | | ATGCTATT | CGGATTAG | AGATGTTA | | |
| | | AGATGGAA | ATGGAAGG | AACAA | | |
| | | GGGAGATT | GAGATTTT | | | |
| | | TTGACAGT | GACAGC | | | |
| 12 | rs12042763 | GAAGGTGA | GAAGGTC | GGAAAAGT | G | T |
| | | CCAAGTTC | GGAGTCAA | ACTGTGAC | | |
| | | ATGCTATG | CGGATTCA | TATTGTGT | | |
| | | AGAGCTAT | TGAGAGCT | ACCAT | | |
| | | AAGTTTTA | ATAAGTTT | | | |
| | | CATGATCA | TACATGAT | | | |
| | | AAC | CAAAA | | | |
| 13 | rs2745559 | GAAGGTGA | GAAGGTC | GTTGTACA | A | C |
| | | CCAAGTTC | GGAGTCAA | AAATTTCT | | |
| | | ATGCTAAG | CGGATTCT | ATTAGCGT | | |
| | | ATTCTTCC | TCCTTATT | CTGAAA | | |
| | | TTATTTTCT | TTCTTTTC | | | |
| | | TTTCATGT | ATGTAGAG | | | |
| | | AGAT | | | | |
| 14 | rs11583191 | GAAGGTGA | GAAGGTC | GAGGGCT | A | G |
| | | CCAAGTTC | GGAGTCAA | GGAGTTG | | |
| | | ATGCTTTA | CGGATTAA | GGTATTTT | | |
| | | ACTGCCTC | CTGCCTCT | TCTT | | |
| | | TAGCTTTC | AGCTTTCC | | | |
| | | CATACAA | ATACAG | | | |
| 15 | rs2143416 | GAAGGTGA | GAAGGTC | CTGCTATA | A | C |
| | | CCAAGTTC | GGAGTCAA | GGACTATA | | |
| | | ATGCTCAA | CGGATTCA | GAATGCTT | | |
| | | TACATTGT | ATACATTG | CACTT | | |
| | | AATGTGAC | TAATGTGA | | | |
| | | GATAAGAGT | CGATAAGA | | | |
| | | | GG | | | |
| 16 | rs2179555 | GAAGGTGA | GAAGGTC | CCTCTGCC | T | C |
| | | CCAAGTTC | GGAGTCAA | ATGTCCAG | | |
| | | ATGCTAAA | CGGATTAC | TCTACTAAT | | |
| | | CACTGGAA | ACTGGAAC | | | |
| | | CAGAAGTC | AGAAGTCA | | | |
| | | AACAATGT | ACAATGC | | | |
| 17 | rs10911907 | GAAGGTGA | GAAGGTC | CTCTTTTTT | A | G |
| | | CCAAGTTC | GGAGTCAA | TCCTTAGC | | |
| | | ATGCTAAA | CGGATTAA | CTGACTAG | | |
| | | AAGGTGGT | GGTGGTTC | AGTA | | |
| | | TCTTTTAA | TTTTAAAA | | | |
| | | AAGACATA | GACATAAA | | | |
| | | AAGTTA | GTTG | | | |
| 18 | rs6681231 | GAAGGTGA | GAAGGTC | GATGGTAC | G | C |
| | | CCAAGTTC | GGAGTCAA | CACATTTT | | |
| | | ATGCTGTG | CGGATTGT | CTTTTACT | | |
| | | ATATATTC | GATATATT | GCTGA | | |
| | | AGTCAGTA | CAGTCAGT | | | |
| | | GAGCATTAG | AGAGCATT | | | |
| | | | AC | | | |

### Selection of Haplotype Tagging SNPs

The software program Haploview (version 3.32) was used for selecting haplotype tagging SNPs. The program implements Tagger pairwise-tagging algorithm, which can directly communicate with HapMap reference datasets, and is available both as an online programme and as stand-alone software. The haplotype tagging SNPs were selected in the PTGS2 gene region, spanning 27 kb, based on SNP genotypes from the HapMap reference CEU dataset. Maximum Entropy analysis was applied for selecting and prioritising the haplotype tagging SNPs.

The tagging thresholds were either chosen at correlation coefficient r²=1.0 and minor allele frequency (maf)≧0.05 or r²≧0.8 and maf≧0.05.

In addition to the selected HapMap haplotype tagging SNPs, six putatively functional SNPs from selected literature were incorporated to define potential functionally important haplotypes, and to establish the patterns of LD between previously studied functional SNPs and HapMap tagging SNPs. These 6 SNPs were rs689469, rs4648298, rs5275, rs20432, rs5277 and rs20417.

The markers rs4648298 and rs689469 are both located in the 3′ untranslated region (UTR) of exon 10, and just upstream and downstream of the last polyadenylation [poly(A)) signal respectively, which is believed to be related to the regulation of transcription. Marker rs4648298 was reported in strong association with increased risk of colorectal cancer in a Spanish case-control study (P=0.01), whereas marker rs689469 showed a marginally significant association with increased risk of colorectal cancer (P=0.05). Marker rs5275 is also located in the 3′ UTR and was suggested to be associated (P=0.04) with risk of lung cancer in a certain age group (50-55 years) in a Danish prospective follow-up study. In contrast, rs20432 in intron 5 was significantly associated with decreased risk of prostate cancer in a large population based case-control study (P=0.02). Marker rs5277 is in exon 3 and protective effects were observed for rs5277 heterozygous in colorectal adenoma patients, although it is a synonymous SNP and does not cause an amino acid change. More promisingly, rs20417 is in a putative SP1 binding site in the promoter and was shown to reduce PTGS2 expression. The homozygous variant (CC) conferred a significant decrease in risk of colorectal adenoma among nonusers of aspirin or other NSAIDs. The medication only reduced risk of adenoma among common variant homozygous (GG) and possibly in heterozygous (GC) patients.

The SNPs selected above were then analysed using the Maximum Entropy method. Entropy was first formulated by Shannon for maximising information content. Recently Aitman et al. proposed that Entropy can serve as a measure of haplotype diversity and be applied to the selection of haplotype tagging SNPs. This method is model free and provides for the selection of optimal haplotype-tagging SNPs even in the absence of a clear haplotypic block structure. Entropy (E) is calculated using the following formula.

$$E = - {\sum }_{i = 1}^{n} {p}_{i} {log}_{2} {p}_{i}$$

where n is the number of different haplotypes in the samples, and pi is the frequency of the ith haplotype. Haplotypes formed by SNPs with larger E values are more diversified and contain more haplotypic information. In principle, those SNPs are treated as haplotype tagging SNPs, or are at least considered as priorities for further analysis. SNPs contributing less Entropy to the haplotypes should generally be considered redundant.

### Analysis of Linkage Disequilibrium and Construction of Haplotype

Based on the data obtained by genotyping the selected haplotype tagging SNPs in a sample population, the degree of linkage disequilibrium (LD) between pairs of SNPs can be analysed. A given SNP is in LD with another SNP when individuals who carry a particular allele at one SNP predictably carry a specific allele at another SNP (usually nearby). Therefore, these two or more SNPs define a block of the genome which is typically inherited as one unit, an LD block.

Numerous software packages are available to analyse LD. For example, the software program Haploview can perform such analysis producing a readout of the LD between any pair of SNPs using the LD coefficient D′ and the square of the correlation coefficient r². The values of both of these measures range from 0 (linkage equilibrium) to 1 (complete linkage disequilibrium). The resulting patterns from such analysis can be used to define the LD blocks in a region, for example using mathematical algorithms such as Gabriel's algorithm, which calculates the LD confidence interval (CI) for each pair of SNPs using a bootstrap method. These LD blocks can then be used to assign an individual a haplotype for an extended region of the genome.

Pair-wise LD for the SNPs identified above was estimated using the LD coefficient D′ and the square of the correlation coefficient r² and, LD blocks were defined. The frequencies of haplotypes and the diplotype configurations (combination of haplotypes assigned to individual subjects) were inferred by the program, PHASE V2.1.1, based on unphased population genotype data. We chose 0.80 as the threshold of confidence level. Therefore, haplotypes were assigned to each individual of 680 VICTOR Trial patients found with a confidence above this threshold.

Association Analyses of Haplotypes with Ages, Tumor Stages and Recurrence

Once the extended haplotypes present in a population have been identified, standard association analysis can be used to identify whether a particular haplotype correlates with a particular phenotype, such as disease susceptibility.

The patterns and frequency distributions of the extended haplotypes display differently in patients with different phenotypes. A patient possessing a particular haplotype will present a different onset of the disease or could respond differently to therapy. Indications of this type provide a basis and guidance for personalised medicine and individualised treatment, improving drug efficacy and reducing or avoiding drug toxicity. For example, a colorectal cancer patient above 65 years of age carrying extended PTGS2 haplotype 1 as identified by the present invention is 2.0 fold more likely to relapse than other patients, with an approximately 70% chance of recurrence, with Odds ratio (OR) 1.7 (95% CI 0.75-3.84). A colorectal cancer patient below 65 years of age carrying extended PTGS2 haplotye 2 as identified by the present invention is 5 fold more likely to relapse than other patients, with an approximately 80% chance of recurrence, with OR 4.5 (95% CI 1.65-12.42).

Association analysis of common haplotypes with age at diagnosis (or age at surgery) of colorectal cancer patients was performed. Tables 2 and 3 summarise the patients analysed. Altogether 680 patients were included in the study, of which 239 were female and 441 male. They are in a range of age 30-86 years with mean age ˜64 and median 65 years. Age 65 was taken as a cut point for dividing patients into two age groups, as the median age is 65 in all. Patients were either in tumour stage II (Duke B) or stage III (Duke C). Among them, 96 patients relapsed by the time of data collected. The patients are further stratified by the haplotypes they carried below.

TABLE 2
Characteristics of Patients (gender and disease stage)

| | | | stage | | |
|---|---|---|---|---|---|
| | | | B (Stage II) | C (Stage III | Total |
| sex | Female | Count | 113 | 126 | 239 |
| | | Expected Count | 114.9 | 124.1 | 239.0 |
| | | % within sex | 47.3% | 52.7% | 100.0% |
| | | % within stage | 34.6% | 35.7% | 35.1% |
| | | % of Total | 16.6% | 18.5% | 35.1% |
| | Male | Count | 214 | 227 | 441 |
| | | Expected Count | 212.1 | 228.9 | 441.0 |
| | | % within sex | 48.5% | 51.5% | 100.0% |
| | | % within stage | 65.4% | 64.3% | 64.9% |
| | | % of Total | 31.5% | 33.4% | 64.9% |
| Total | | Count | 327 | 353 | 680 |
| | | Expected Count | 327.0 | 353.0 | 680.0 |
| | | % within sex | 48.1% | 51.9% | 100.0% |
| | | % within stage | 100.0% | 100.0% | 100.0% |
| | | % of Total | 48.1% | 51.9% | 100.0% |

TABLE 3
Characteristics of Relapsed Patients (gender and disease stage)

| stage | gender | N | Mean | Median |
|---|---|---|---|---|
| B (Stage II) | Female | 10 | 60.11 | 57.29 |
| | Male | 20 | 66.85 | 68.54 |
| | Total | 30 | 64.60 | 67.46 |
| C (Stage III | Female | 22 | 59.95 | 60.25 |
| | Male | 44 | 63.90 | 64.21 |
| | Total | 66 | 62.59 | 62.13 |
| Total | Female | 32 | 60.00 | 59.21 |
| | Male | 64 | 64.82 | 64.46 |
| | Total | 96 | 63.22 | 63.96 |

### Results

### Selection of Tagging SNPs and Allele Frequencies in the Study Cohort

A total number of 81 SNPs were found from the HapMap CEU reference dataset in the PTGS2 region covering about 27 kb, at an average density of 3.0 SNPs/kb. A panel of 11 tagging SNPs were selected, with tagging threshold of correlation coefficient r²=1.0 and maf≧0.05. The panel of tagging SNPs captured variation at 24 loci with mean r²=1.0, which represents 30% of all SNPs in the region. With less stringent tagging threshold (r²≧0.8, maf≧0.05), a subset of 7 tagging SNPs was defined. In addition, 6 putatively functional SNPs were selected from literature. These were rs689469, rs4648298, rs5275, rs20432, rs5277 and rs20417. The marker rs5272, was also included as it had previously selected as a haplotype tagging SNP based on an earlier release of the HapMap dataset (Release #20 Jan. 2006). However, it has since been shown to be non-variant in Caucasian populations. The total number of SNPs selected was therefore 18.

All 18 selected SNPs (Table 4) were genotyped in the VICTOR Trial patients (n=706). The flanking sequences of the SNPs are shown in Table 5. The minor allele frequency distribution is shown in Table 6. Most SNPs selected are common polymorphisms. Among them, 13 SNPs (72% of 18 SNPs) have a minor allele frequency (maf)>10%, with rs5275 having the highest maf (32.8%).

### Analysis of Linkage Disequilibrium and Haplotype

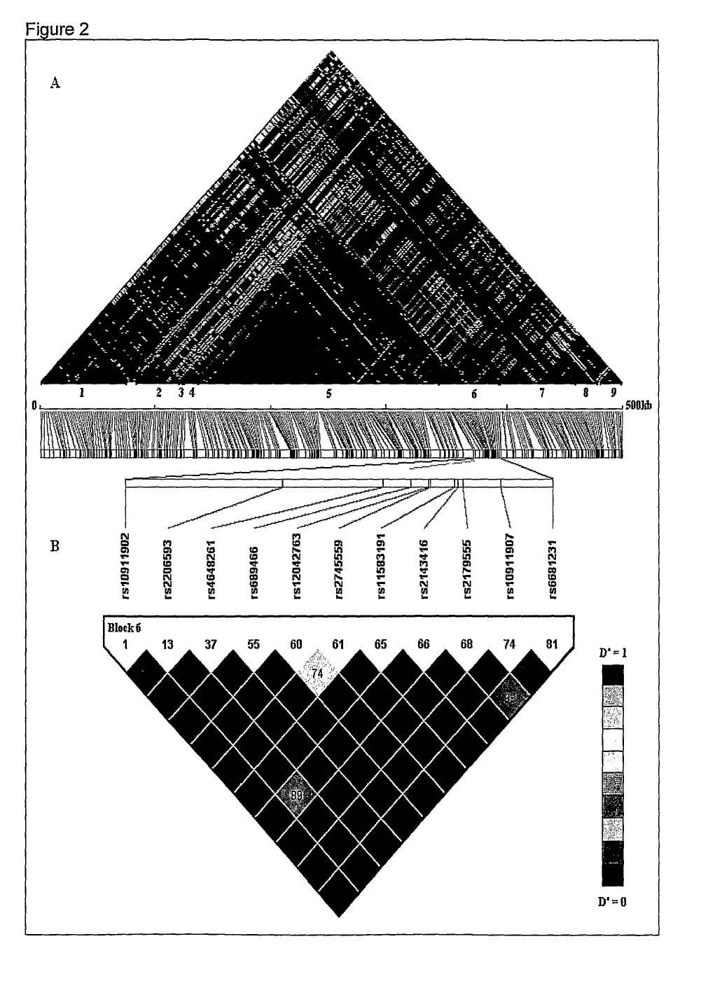

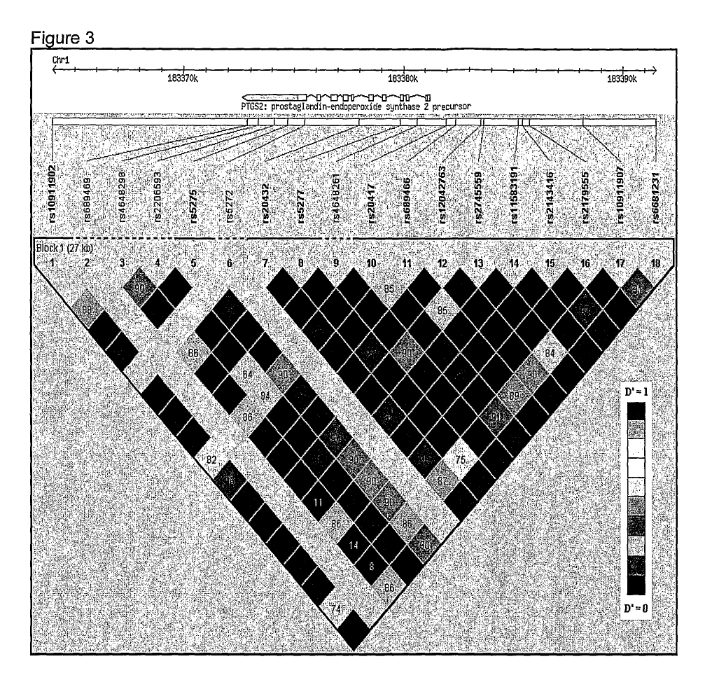

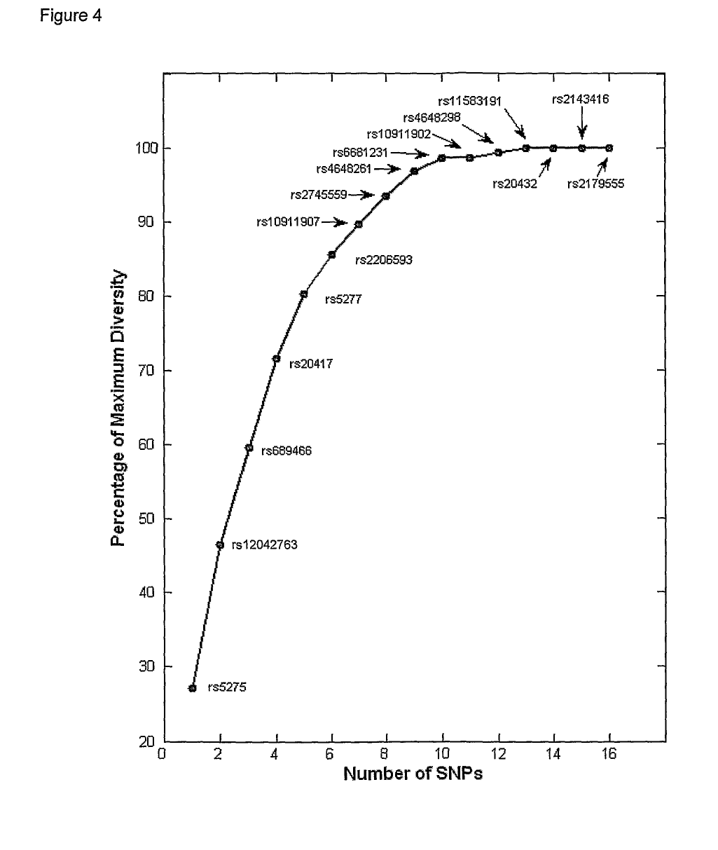

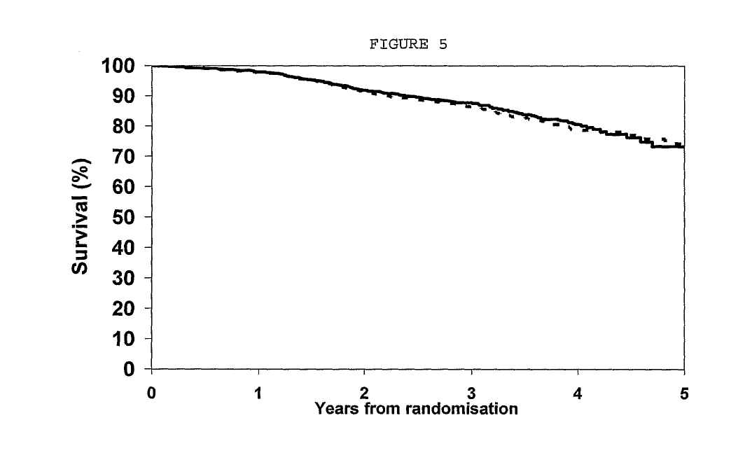

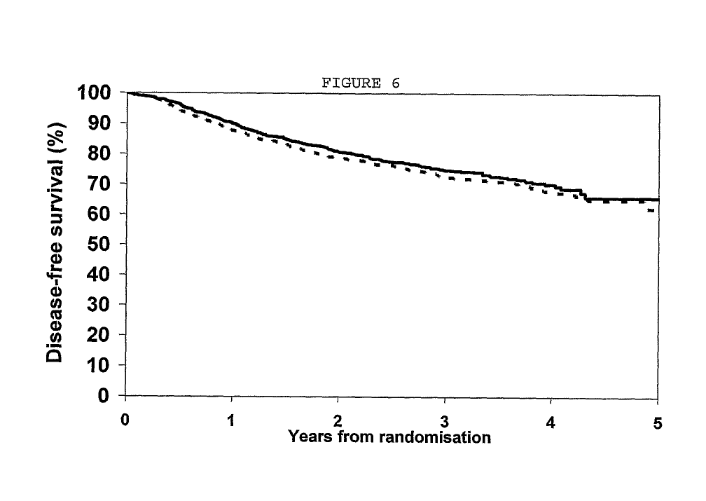

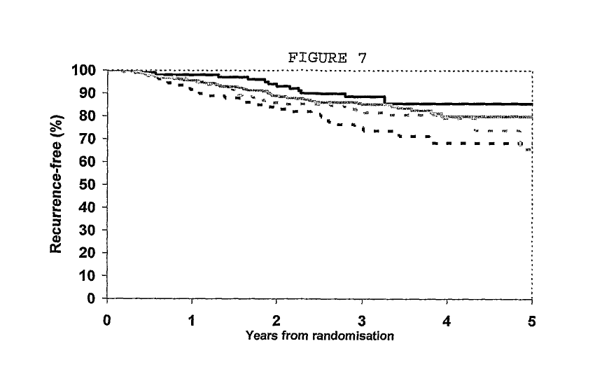

Based on the HapMap CEU dataset, 9 LD blocks were found within the extended PTGS2 gene region, spanning 500 kb (FIG. 2). The gene of interest is located in block 6, the fourth largest block in the extended region. We then examined closely the ˜30 kb gene region. This block can be defined by 11 tagging SNPs (at r²=1 and maf≧0.05). FIG. 2 panel B illustrates pair-wise D′ value for all 11 tagging SNPs, which shows that most of them were in complete LD (D′=1.0) and three pairs of SNPs were in strong LD (12042763/rs2745559 (D′=0.74), rs2206593/rs2143416 (D′=0.89), and rs2179555/rs6681231 (D′=0.93)).

TABLE 4
Locations and minor allele frequencies of the selected SNPs in PTGS2 region

| | dbSNP ID | Position | Region | MA | MAF | Panel A | Panel B | Panel C |
|---|---|---|---|---|---|---|---|---|
| 1 | rs10911902 | 183363974 | 3′ | T | 0.200 | + | + | − |
| 2 | rs689469 | 183372854 | 3′ UTR | A | 0.022 | − | − | + |
| 3 | rs4648298 | 183373339 | 3′ UTR | G | 0.013 | − | − | + |
| 4 | rs2206593 | 183374086 | 3′ UTR | A | 0.058 | + | + | − |
| 5 | rs5275 | 183374715 | 3′ UTR | C | 0.312 | − | − | + |
| 6 | rs5272 | 183375494 | Exon 10 | G | 0.217 | − | − | + |
| 7 | rs20432 | 183377980 | Intron 5 | G | 0.196 | − | − | + |
| 8 | rs5277 | 183379854 | Exon 3 | C | 0.239 | − | − | + |
| 9 | rs4648261 | 183380661 | Intron 1 | A | 0.058 | + | + | − |
| 10 | rs20417 | 183381978 | Promoter | C | 0.238 | − | − | + |
| 11 | rs689466 | 183382408 | Promoter | G | 0.125 | + | + | − |
| 12 | rs12042763 | 183383533 | Promoter | T | 0.280 | + | + | − |
| 13 | rs2745559 | 183383659 | 5′ | A | 0.136 | + | + | − |
| 14 | rs11583191 | 183385207 | 5′ | A | 0.155 | + | − | − |
| 15 | rs2143416 | 183385422 | 5′ | C | 0.184 | + | − | − |
| 16 | rs2179555 | 183385733 | 5′ | C | 0.164 | + | − | − |
| 17 | rs10911907 | 183388159 | 5′ | G | 0.151 | + | − | − |
| 18 | rs6681231 | 183391516 | 5′ | C | 0.175 | + | + | − |

MA: minor allele;
MAF: minor allele frequency;
+ Selected,
− None-selected.
* rs5272 was previously selected as tagging SNP using older release of HapMap CEU dataset Release #20 January 2006.
Panel A: Tagging SNPs selected at r² = 1.0 and MAF ≧ 0.05.
Panel B: Tagging SNPs selected at r² = 0.8 and MAF ≧ 0.05.
Panel C: Putatively functional SNPs selected from literature.

TABLE 5

Flanking sequences of the selected SNPs in the
PTGS2 region

| | | | |
|---|---|---|---|
| SNP ID | dbSNP ID | SEQ ID | Sequence |

| | | | |
|---|---|---|---|
| 1 | rs10911902 | 2 | CCCCAACACACAAAAACAATTATAACAAAAAT |
| | | | AAACATATTCTGGCTCTTAATGGGTGGTCCTA |
| | | | TGTTTGTGATCAGTGT [Y] AGTGATTAGTGCAGA |
| | | | TTCTAGGACTATTTTTTTTTTCCTTTCATA |
| | | | ACATGCAGTGAAAA |
| 2 | rs689469 | 3 | CTTTTGGGAAGAGGGAGAAAATGAAATAAATA |
| | | | TCATTAAAGATAACTCAGGAGAATCTTCTTTA |
| | | | CAATTTTAC [R] TTTAGAATGTTTAAGGTTAA |
| | | | GAAAGAAATAGTCAATATGCTTGTATAAAACA |
| | | | CTGTTCACTGTTT |
| 3 | rs4648298 | 4 | CTGACATTTAATGGTACTGTATATTACTTAAT |
| | | | TTATTGAAGATTATTATTTATGTCTTATTAGG |
| | | | ACACTATGGTTATAA [R] CTGTGTTTAAGCCT |
| | | | ACAATCATTGATTTTTTTTTGTTATGTCACAA |
| | | | TCAGTATATTTTCT |
| 4 | rs2206593 | 5 | TTATGAGGTCATTGCTACTTTTGCAATGTGAT |
| | | | ATGGACTGCTAAATTAAACTGTACAACAGAAA |
| | | | ATCTGAGAAAACATATC [R] TTATTCAAGCAC |
| | | | AGCTTGGTACTTCATTAACCTCATAGCAAAAT |
| | | | CTGAGTACCAGGCC |
| 5 | rs5275 | 6 | TTACACTGTCGATGTTTCCAATGCATCTTCCA |
| | | | TGATGCATTAGAAGTAACTAATGTTTGAAATT |
| | | | TTAAAGTACTTTTGGT [Y] ATTTTTCTGTCAT |
| | | | CAAACAAAARCAGGTATCAGTGCATTATTAAA |
| | | | TGAATATTTAAATT |
| 6 | rs5272 | 7 | TCTTCATCGCCTTCACAGGAGAAAAGGAAATG |
| | | | TCTGCAGAGTTGGAAGCACTCTATGGTGACAT |
| | | | CGATGCTGTGG [R] GCTGTATCCTGCCCTTCT |
| | | | GGTAGAAAAGCCTCGGCCAGATGCCATCTTTG |
| | | | GTGAAACCATG |
| 7 | rs20432 | 8 | TATTTTTTGGATTTCAATAGCATAGCTTCAAG |
| | | | TTATTCGTAAGAATTTTTTATAAATAATACAT |
| | | | TTTTATACTTTTTTA [K] AATTACCATATCAT |
| | | | CATAGTGAAGTATATAATATATATGATATAAG |
| | | | CTCAATATAGTATA |
| 8 | rs5277 | 9 | ACATACTTACCCACTTCAAGGGATTTTGGAAC |
| | | | GTTGTGAATAACATTCCCTTCCTTCGAAATGC |
| | | | AATTATGAGTTATGT [S] TTGACATGTAAGTA |
| | | | CAAGTGTCTTTCTAAGGTTTTTAGCCTTCTCA |
| | | | AAGAAAAATATGCT |
| 9 | rs4648261 | 10 | CAGGATCTGATCAATATATGTGAATTGTTTAT |
| | | | ATTTGGAACCTTTTTATTGAGTGGAAGAAGTT |
| | | | GTTTTAAATATTCTA [R] TCAGTTCTTTCCTG |
| | | | CTCCCAGGAAAGCCCGGATTATGTTTTAAGAT |
| | | | AAGCAAAATGTCTT |
| 10 | rs20417 | 11 | TGGTGACCCGTGGAGCTCACATTAACTATTTA |
| | | | CAGGGTAACTGCTTAGGACCAGTATTATGAGG |
| | | | AGAATTTACCTTTCCC [S] CCTCTCTTTCCAA |
| | | | GAAACAAGGAGGGGGTGAAGGTACGGAGAACA |
| | | | GTATTTCTTCTGTT |
| 11 | rs689466 | 12 | TTTAGTATCTCACCCTCACATGCTCCTCCCTG |
| | | | AGCACTACCCATGATAGATGTTAAACAAAAGC |
| | | | AAAGATGAAATTCCA [R] CTGTCAAAATCTCC |
| | | | CTTCCATCTAATTAATTCCTCATCCAACTATG |
| | | | TTCCAAAACGAGAA |
| 12 | rs12042763 | 13 | AATGTGCCAAACTAAATTAAGACCACTAAACC |
| | | | TGTTTTATATGGAAAAGTACTGTGACTATTGT |
| | | | GTACCATAAAAAAAAG [K] TTTGATCATGTAA |
| | | | AACTTATAGCTCTCATGTTTGCATATATTCAA |
| | | | TGTTTCTGTGTCTA |
| 13 | rs2745559 | 14 | TGTTTCTGTGTCTATTTTAGGCAAATATATGG |
| | | | ATCTGTTGTACAAAATTTCTATTAGCGTCTGA |
| | | | AATTTGTTGGGAAATA [M] TCTACATGAAAAG |
| | | | AAAATAAGGAAGAATCTTAAAAATCTTCCAGA |
| | | | GCTTTCTGTTGAGT |
| 14 | rs11583191 | 15 | CTGTCGGGATTTTTGTCGCAGCCTAACTGAAT |
| | | | TAGAAGAAGGAAAATATCCAAATTTAACTGCC |
| | | | TCTAGCTTTCCATACA [R] GGGAAGAAAAATA |
| | | | CCCAACTCCAGCCCTCTCTTTAGCTTCTACAT |
| | | | TGGAGAAGGGAAAT |
| 15 | rs2143416 | 16 | GAAAGTCACCGTTCATAGGCACAGGCTCACTA |
| | | | AAAGGCCGAGACCTGCTATAGGACTATAGAAT |
| | | | GCTTCACTTCCCCTGA [M] CTCTTATCGTCAC |
| | | | ATTACAATGTATTGAGTTTCTTTCACTCAGTA |
| | | | CATCATGCCTGGCT |
| 16 | rs2179555 | 17 | GTAAGTAGACAGATGGCAATTCCAAGAAATAA |
| | | | TCAGAGAAATGCTAGAGATCAAAAACACTGGA |
| | | | ACAGAAGTCAACAATG [Y] CTGTAAGGGCTTA |
| | | | TTAGTAGACTGGACATGGCAGAGGAAAGAATC |
| | | | CCTGCGATTGCAGA |
| 17 | rs10911907 | 18 | TCAATCAAATCAAAAACAGAAAATCAAAAAAG |
| | | | AAAATGGGTAAAACCAAAAAGGTGGTTCTTTT |
| | | | AAAAGACATAAAGTT [R] ATTACTCTAGTCAG |
| | | | GCTAAGGAAAAAAAGAGAAGACACAAATTATT |
| | | | AGTATCAGAATTGA |
| 18 | rs6681231 | 19 | TCAAAAACTTGGAAACAATCAAGATGTACTTC |
| | | | AGAAGGAGAACGGATTAATTGTGATATATTCA |
| | | | GTCAGTAGAGCATTA [S] TCAGCAGTAAAAGA |
| | | | AAATGTGGTACCATCATATATTTAAGACATGG |
| | | | AGGAATCTTAAATA |

TABLE 6
Minor allele frequencies of 18 SNPs in VICTOR cohort

| SNP ID | N | MAF (n) |
|---|---|---|
| rs10911902 | 1390 | 0.187 (260) |
| rs689469 | 1322 | 0 (0) |
| rs4648298 | 1396 | 0.014 (20) |
| rs2206593 | 1360 | 0.049 (66) |
| rs5275 | 1380 | 0.328 (452) |
| rs5272 | 1390 | 0 (0) |
| rs20432 | 1358 | 0.139 (189) |
| rs5277 | 1380 | 0.149 (205) |
| rs4648261 | 1384 | 0.032 (44) |
| rs20417 | 1364 | 0.152 (207) |
| rs689466 | 1310 | 0.198 (259) |
| rs12042763 | 1348 | 0.256 (345) |
| rs2745559 | 1364 | 0.18 (245) |
| rs11583191 | 1390 | 0.132 (183) |
| rs2143416 | 1376 | 0.146 (201) |
| rs2179555 | 1378 | 0.131 (180) |
| rs10911907 | 1354 | 0.145 (197) |
| rs6681231 | 1386 | 0.147 (204) |

N: number of chromosomes genotyped;
n: number of minor alleles detected

Pair-wise D′ values were also analysed for all pairs of 18 SNPs genotyped in the VICTOR Trial patients (FIG. 3). A single LD block was defined. All SNPs are in complete and strong LD except SNP marker rs4648298, which is in weak LD with markers rs11583191, rs2179555 and rs10911907. Two SNPs (rs5272 and rs689469) were invariant in the VICTOR Trial patients. Haplotypes were constructed using the PHASE program based on 16 SNPs (excluding two invariant SNPs). Although 66 haplotypes were inferred using the program based on 706 VICTOR samples in total (data not shown), only 27 of them were found in 680 patients with haplotype confidence level above the threshold of 0.80 (Table 7). Ten haplotypes had an individual haplotype frequency of above 1.0%, and account for 97% of the patient population. The first three common haplotypes represent 48.8% of the population, with a single haplotype (Hap 1, the most common one) alone accounting for about 20%. There are four haplotypes, each of which can be tagged by a single minor allele (MA) of a SNP. They are Hap 1 tagged by the allele ‘G’ of rs689466, Hap 6 by the allele ‘T’ of rs12042763, Hap 12 by the allele ‘C’ of rs5277 and Hap 13 by the allele ‘C’ of rs5275. Representing less than 2% of the patient population, Hap 9 is defined by all major alleles from those 16 SNPs. These findings are summarised in Table 7.

### Analysis of Entropy

The Entropy Maximisation Method was adopted for prioritising Tagging SNPs and detecting the maximum haplotypic diversity. The entropies were calculated for the 16 SNPs which varied in this cohort. In this analysis, the maximum haplotypic diversity (E=1.00746) was captured by the haplotype composed of 13 SNPs. These SNPs were rs5275, rs12042763, rs689466, rs20417, rs5277, rs2206593, rs10911907, rs2745559, rs4648261, rs6681231, rs10911902, rs4648298 and rs11583191. These findings are summarised in FIG. 4. The first 6 SNPs account for about 85% of the haplotypic diversity, and the first 10 SNPs account for nearly 97%. The remaining 6 SNPs only contribute 3%.

Association Analyses of Haplotypes with Ages at Diagnosis

The VICTOR Trial patients were divided into two major groups according to their age at diagnosis or at surgery, either younger or older than 65 years, and further stratified by the common haplotypes, as shown in Table 8. There was no significant association detected between common haplotypes and two major age groups (p=0.6884 in Table 8).

TABLE 7
Patterns and Frequencies of Haplotypes in VICTOR Trial patients
In the title row, each SNP is identified by SNP ID no. (from Table 5) and the identity
of its major allele is indicated. In the rows for each haplotype (Hap ID 1-27), the
minor alleles for a given SNP are indicated where they characterise a given
haplotype. “—” indicates that the major allele is present.

| | SNPs | | | | | | | | | | | | | | | | | |
|---|---|---|---|---|---|---|---|---|---|---|---|---|---|---|---|---|---|---|
| Hap | 1 | 3 | 4 | 5 | 7 | 8 | 9 | 10 | 11 | 12 | 13 | 14 | 15 | 16 | 17 | 18 | Frequency | |
| ID | C | A | G | T | T | G | G | G | A | G | C | G | A | T | A | G | n | % |
| 1 | — | — | — | — | — | — | — | — | G | — | — | — | — | — | — | — | 263 | 19.95 |
| 2 | T | — | — | C | — | — | — | — | — | — | — | — | — | — | — | — | 190 | 14.42 |
| 3 | — | — | — | — | — | C | — | — | — | T | — | — | — | — | — | — | 190 | 14.42 |
| 4 | — | — | — | — | — | — | — | — | — | — | A | — | — | — | — | — | 169 | 12.82 |
| 5 | — | — | — | C | G | — | — | C | — | — | — | A | C | C | G | C | 165 | 12.52 |
| 6 | — | — | — | — | — | — | — | — | — | T | — | — | — | — | — | — | 151 | 11.46 |
| 7 | — | — | A | — | — | — | — | — | — | — | A | — | — | — | — | — | 61 | 4.63 |
| 8 | T | — | — | C | — | — | A | — | — | — | — | — | — | — | — | — | 43 | 3.26 |
| 9 | — | — | — | — | — | — | — | — | — | — | — | — | — | — | — | — | 24 | 1.82 |
| 10 | — | G | — | C | G | — | — | C | — | — | — | — | C | — | — | C | 16 | 1.21 |
| 11 | — | — | — | — | — | — | — | C | — | — | — | A | C | C | G | C | 8 | 0.61 |
| 12 | — | — | — | — | — | C | — | — | — | — | — | — | — | — | — | — | 7 | 0.53 |
| 13 | — | — | — | C | — | — | — | — | — | — | — | — | — | — | — | — | 5 | 0.38 |
| 14 | T | — | — | C | — | — | — | — | — | — | — | — | — | — | G | — | 4 | 0.3 |
| 15 | — | — | — | — | G | — | — | C | — | — | — | A | C | C | G | C | 4 | 0.3 |
| 16 | — | — | — | — | — | — | — | — | G | — | A | — | — | — | — | — | 3 | 0.23 |
| 17 | — | — | — | — | — | — | — | — | G | — | — | — | — | — | G | — | 2 | 0.15 |
| 18 | — | — | — | — | — | C | — | — | — | T | — | — | — | — | G | — | 2 | 0.15 |
| 19 | — | — | — | C | G | — | — | C | — | — | — | — | C | — | — | C | 2 | 0.15 |
| 20 | T | G | — | C | — | — | — | — | — | — | — | — | — | — | — | — | 2 | 0.15 |
| 21 | — | — | — | — | — | — | — | C | G | — | — | — | — | — | — | — | 1 | 0.08 |
| 22 | — | — | — | C | — | — | — | — | — | — | A | — | — | — | G | — | 1 | 0.08 |
| 23 | — | — | — | C | G | — | — | — | — | — | — | A | C | C | G | C | 1 | 0.08 |
| 24 | T | — | — | C | — | — | — | C | — | — | — | — | — | — | G | — | 1 | 0.08 |
| 25 | T | — | — | C | — | — | — | — | G | — | — | — | — | — | — | — | 1 | 0.08 |
| 26 | — | — | — | C | — | — | — | — | — | — | A | — | — | — | — | — | 1 | 0.08 |
| 27 | — | — | — | — | — | — | — | — | G | — | — | — | C | — | — | — | 1 | 0.08 |

| | | |
|---|---|---|
| Total | 1318 | 100 |

n: number of chromosomes observed in VICTOR patient samples with confidence level ≧0.8.

TABLE 8
Frequency distribution of common haplotype in Age groups

| | <65 years | Others | Sub | total |
|---|---|---|---|---|
| Hap ID | (n) | (n) | (n) | (%) |
| 1 | 138 | 130 | 268 | 19.71 |
| 2 | 105 | 92 | 197 | 14.49 |
| 3 | 100 | 98 | 198 | 14.56 |
| 4 | 91 | 89 | 180 | 13.24 |
| 5 | 79 | 86 | 165 | 12.13 |
| 6 | 81 | 67 | 148 | 10.88 |
| 7 | 29 | 34 | 63 | 4.63 |
| 8 | 28 | 16 | 44 | 3.24 |
| 9 | 11 | 12 | 23 | 1.69 |
| 10 | 11 | 6 | 17 | 1.25 |
| others | 33 | 24 | 57 | 4.19 |
| Total | 706 | 654 | 1360 | 100.00 |

P = 0.6884

Association Analyses of Haplotypes with Tumor Stages

The tumor stages in patients were stratified by their common haplotypes. Over all, there is no significant difference detected (p=0.2433 in Table 9). However, the distributions of some haplotype groups (or haplotype clusters) show significant difference between stages (Table 10). By “haplotype group”, it is intended to refer to both of the haplotypes present in an individual, i.e. the extended PTGS2 haplotype present on both copies of chromosome 1 present in said individual. For example, haplotype group “1,4” indicates one chromosome is of haplotype 1 and the other is of haplotype 4. The p values for extended PTGS2 haplotype groups ‘1,4’; ‘1,5’; ‘1,9’ and ‘2,6’ are 0.0176, 0.0112, 0.0335 and 0.0350 respectively.

Haplotypes are also differently distributed between two age groups of patients in tumour stage III (p=0.0085 in Table 11). Except haplotype group ‘2,5’ (p=0.35342), other haplotype groups show marginal or significant differences, with the lowest p value for haplotype group ‘3,5’ (p=0.00012, see Table 12).

TABLE 9
Frequency distribution of common haplotypes in tumor stages

| | Stage II | Stage III | Sub | total |
|---|---|---|---|---|
| Hap ID | (n) | (n) | (n) | (%) |
| 1 | 143 | 125 | 268 | 19.71 |
| 2 | 89 | 108 | 197 | 14.49 |
| 3 | 92 | 106 | 198 | 14.56 |
| 4 | 93 | 87 | 180 | 13.24 |
| 5 | 87 | 78 | 165 | 12.13 |
| 6 | 60 | 88 | 148 | 10.88 |
| 7 | 26 | 37 | 63 | 4.63 |
| 8 | 19 | 25 | 44 | 3.24 |
| 9 | 13 | 10 | 23 | 1.69 |
| 10 | 8 | 9 | 17 | 1.25 |
| others | 24 | 33 | 57 | 4.19 |
| Total | 654 | 706 | 1360 | 100.00 |

p = 0.2433

TABLE 10
Association analysis of common haplotype groups with tumor stages

| | Hap group | | | | | | | | |
|---|---|---|---|---|---|---|---|---|---|
| | 1,2 | 1,3 | 1,4 | 1,5 | 1,6 | 1,7 | 1,8 | 1,9 | 1,10 |
| P value | 0.3371 | 0.2122 | 0.0176 | 0.0112 | 0.7280 | 0.2139 | 0.1225 | 0.0335 | 0.062882 |
| | Hap group | | | | | | | | |
| | 1,2 | 2,3 | 2,4 | 2,5 | 2,6 | 2,7 | 2,8 | 2,9 | 2,10 |
| P value | 0.3371 | 0.2847 | 0.9317 | 0.8136 | 0.0350 | 0.1663 | 0.2619 | 0.5760 | 0.3785 |

Summarising this result: as an example, haplotype groups 1,5; 1,4 and 1,9 (top panel of table 10) are associated with tumor stages (between stage II and III) in the patient population (p=0.0112 for 1,5). Therefore, statistically patients carrying these haplotypes are more likely to present in stage II. Those patients are likely to benefit from chemotherapy and have better prognosis. In contrast, patients with haplotype group 2,6 (bottom panel of table 10) are more likely to present in stage III, and are likely to have a worse prognosis.

TABLE 11
Frequency distribution of common haplotypes in two age groups of
stage III patients.

| | <65 | Others | Sub | total |
|---|---|---|---|---|
| Hap ID | (n) | (n) | (n) | (%) |
| 1 | 74 | 51 | 125 | 17.71 |
| 2 | 69 | 39 | 108 | 15.30 |
| 3 | 51 | 55 | 106 | 15.01 |
| 4 | 52 | 35 | 87 | 12.32 |
| 5 | 31 | 47 | 78 | 11.05 |
| 6 | 53 | 35 | 88 | 12.46 |
| 7 | 19 | 18 | 37 | 5.24 |
| 8 | 17 | 8 | 25 | 3.54 |
| 9 | 4 | 6 | 10 | 1.42 |
| 10 | 8 | 1 | 9 | 1.27 |
| others | 22 | 11 | 33 | 4.67 |
| Total | 400 | 306 | 706 | 100.00 |

P = 0.0085

TABLE 12
Association analysis of common haplotype groups with age of stage III patients

| | Hap group | | | | | | | | |
|---|---|---|---|---|---|---|---|---|---|
| | 1,5 | 2,5 | 3,5 | 4,5 | 5,6 | 5,7 | 5,8 | 5,9 | 5,10 |
| P value | 0.0929 | 0.35342 | 0.00012 | 0.05989 | 0.07186 | 0.00183 | 0.02586 | 0.00063 | 0.01741 |

Thus, patients carrying haplotype group 3,5; 5,7; 5,8; 5, 9 or 5,10 are more likely to present in older age (>65 years) and with stage III.
Association Analyses of Haplotypes with Recurrence

No significant difference was detected among common haplotype distributions between relapsed and non-relapsed patients (p=0.6535 in Table 13). However, significant difference was found between the two age group in relapsed patients (p=0.0215 in Table 14). In particular, the difference was substantially contributed by single haplotype, i.e. haplotype 1 (p=0.0021) and haplotype 2 (p=0.0017) (Table 15). Except haplotype group ‘1,2’, most other haplotype groups show a significant difference, with the lowest p values for haplotype group ‘2,10’ (p=0.0007) and haplotype group ‘1,9’ (p=0.0012) (Table 15).

TABLE 13
Frequency distribution of common haplotypes in relapsed and non-
relapsed patients.

| | Recur | Non-recur | Sub | total |
|---|---|---|---|---|
| Hap ID | (n) | (n) | (n) | (%) |
| 1 | 31 | 237 | 268 | 19.71 |
| 2 | 30 | 167 | 197 | 14.49 |
| 3 | 30 | 168 | 198 | 14.56 |
| 4 | 22 | 158 | 180 | 13.24 |
| 5 | 28 | 137 | 165 | 12.13 |
| 6 | 23 | 125 | 148 | 10.88 |
| 7 | 7 | 56 | 63 | 4.63 |
| 8 | 5 | 39 | 44 | 3.24 |
| 9 | 3 | 20 | 23 | 1.69 |
| 10 | 5 | 12 | 17 | 1.25 |
| others | 8 | 49 | 57 | 4.19 |
| Total | 192 | 1168 | 1360 | 100.00 |

P = 0.6535

TABLE 14
Frequency distribution of common haplotypes in two major groups of
relapsed patients

| | <65 | Others | Sub | total |
|---|---|---|---|---|
| Hap ID | (n) | (n) | (n) | (%) |
| 1 | 10 | 21 | 31 | 16.15 |
| 2 | 25 | 5 | 30 | 15.63 |
| 3 | 17 | 13 | 30 | 15.63 |
| 4 | 11 | 11 | 22 | 11.46 |
| 5 | 18 | 10 | 28 | 14.58 |
| 6 | 12 | 11 | 23 | 11.98 |
| 7 | 3 | 4 | 7 | 3.65 |
| 8 | 3 | 2 | 5 | 2.60 |
| 9 | 1 | 2 | 3 | 1.56 |
| 10 | 4 | 1 | 5 | 2.60 |
| others | 6 | 2 | 8 | 4.17 |
| Total | 110 | 82 | 192 | 100.00 |

P = 0.0215

TABLE 15
Association analysis of common haplotypes with age in relapsed patients

| | Hap group | | | | | | | | | |
|---|---|---|---|---|---|---|---|---|---|---|
| | 1 | 1,2 | 1,3 | 1,4 | 1,5 | 1,6 | 1,7 | 1,8 | 1,9 | 1,10 |
| P value | 0.0021 | 0.9870 | 0.0128 | 0.0022 | 0.0665 | 0.0037 | 0.0013 | 0.0044 | 0.0012 | 0.0133 |
| | Hap group | | | | | | | | | |
| | 2 | 1,2 | 2,3 | 2,4 | 2,5 | 2,6 | 2,7 | 2,8 | 2,9 | 2,10 |
| P value | 0.0017 | 0.9870 | 0.0164 | 0.0415 | 0.0019 | 0.0303 | 0.0119 | 0.0027 | 0.0061 | 0.0007 |

Thus, patients carrying haplotype 1 (or any other underlined haplotype group in the top panel of Table 15) and of younger age (>65 years) have less chance of relapse (top panel). Patients with haplotype 2 (or any other underlined haplotype group in the bottom panel) and of younger age (>65 years) have more chance of relapse.

### CONCLUSIONS

The above analyses show:

That a colorectal cancer patient with haplotype group ‘1,4’; ‘1,5’ or ‘1,9’ is less likely to progress to Stage III. In contrast, a colorectal cancer patient with haplotype group 2,6 is more likely to progress to Stage III, if age is not adjusted.

That a colorectal cancer patient above 65 years of age with haplotype group ‘3,5’; ‘5,7’; ‘5,8’; ‘5,9’ or ‘5,10’ is more likely to progress to Stage III.

That a colorectal cancer patient above 65 years of age carrying haplotype 1 is 2.0 fold more likely to relapse than other patients, with an approximately 70% chance of recurrence, with Odds ratio (OR) 1.7 (95% CI 0.75-3.84).

That a colorectal cancer patient below 65 years of age carrying haplotype 2 is 5 fold more likely to relapse than other patients, with an approximately 80% chance of recurrence, with OR 4.5 (95% CI 1.65-12.42).

Indications of this type provide a basis and guidance for future personalised medicine and individualised treatment.

Genetic variations in PTGS2 play an important role in the development and progression of cancers. Therefore, it is necessary to fully understand the architecture of the gene and the relationships among its genetic variants, not only in general populations but also in patient populations. In our study, we have examined the genetic structure of the extended gene region, and identified tagging SNPs which can be used to depict molecular patterns, i.e. LD blocks and haplotype structures, in colorectal cancer patient population.

HapMap reference datasets provide useful resources for study of normal healthy populations. We have successfully applied these resources as guidance to selecting minimum tagging SNPs for our analysis on clinical datasets. In this study, we analysed patterns of LD blocks and haplotype structures in PTGS2 region using the dataset from the HapMap CEU population. By examining the extended 500 kb region covering PTGS2, we found that this 500 kb region was just between two adjacent recombination hotspots and had little evidence for historical recommendation. In general, the region is a homogeneous block in strong LD. For comparison, we also analysed the region in the Chinese population (CHB) and African population (YRI). A similar pattern was also seen in the Chinese population (CHB). In contrast, a heterogeneous block in the African population (YRI) has been found. It is well accepted that the genetic variability and complexity is in compliance with the human evolution, demographic history and geographic localisation, which may imply the origins of human diseases.

The less complex genetic structure in the gene region indicates that a small number of SNPs or even a single SNP can mark a longer stretch of haplotype, e.g in the European and Chinese populations. However, more SNPs may be demanded to mark even a short stretch of haplotype, e.g. in African populations. For instance, a minimum of 7 tagging SNPs (with their minor allele frequencies above 5% at r2≧0.8) were needed to capture most of the haplotypic information in PTGS2 region (spanning ˜30 kb) in the European population (CEU), while only 4 tagging SNPs were needed in the Chinese population (CHB). Conversely at least 16 tagging SNPs should be genotyped in the African population (YR1) to capture a similar amount of information.

In addition to tagging SNPs from the HapMap resource, we also chose 6 SNPs, rs689469, rs4648298, rs5275, rs20432, rs5277 and rs20417 from the literature. The markers rs4648298 and rs689469 are both located in the 3′ untranslated region (UTR) of exon 10, and just nearby upstream and downstream of the last polyadenylation [poly(A)) signal respectively, which is believed to be related to the regulation of transcription. The former marker (rs4648298) was reported in strong association with increased risk of colorectal cancer in a Spanish case-control study (P=0.01), whereas the latter (rs689469) showed a marginally significant association with increased risk of colorectal cancer (P=0.05). Marker rs5275 is also located in the 3′ UTR and was suggested to be associated (P=0.04) with risk of lung cancer in a certain age group (50-55 years) in a Danish prospective follow-up study. In contrast, rs20432 in intron 5 was significantly associated with decreased risk of prostate cancer in a large population based case-control study (P=0.02). Marker rs5277 is in exon 3 and protective effects were observed for rs5277 heterozygous in colorectal adenoma patients, although it is a synonymous SNP and does not cause an amino acid change. More promisingly, rs20417 is in a putative SP1 binding site in the promoter and was shown to reduce PTGS2 expression. The homozygous variant (CC) conferred a significant decrease in risk of colorectal adenoma among nonusers of aspirin or other NSAIDs. The medication only reduced risk of adenoma among common variant homozygous (GG) and possibly in heterozygous (GC) patients. In our analysis, one of these SNP (rs5275) was favoured by Entropy analysis, and nominated as the most informative SNP among all SNPs. This marker alone can capture 27% of the diversity. Combining SNPs rs12042763 and rs689466 with rs5275, it could capture 47% and 60% of the diversity respectively.

Based on 16 SNPs, a total of 27 different haplotypes representing 1318 chromosomes were found with confidence levels of above 0.8 in our patient samples. We expected to find that a few tagging SNPs could denote most of the haplotypes. In fact, we found 4 out of 27 haplotypes can be denoted by a single SNP, and represent ˜45% of the total chromosomes in the patient population. For example, hap1 is denoted by a single tagging SNP rs689466, which is the most common haplotype and represents ˜20% of the total chromosomes. The 6 most common haplotypes (>10%) accounted for more than 85% of the chromosomes. Therefore, our study results support the idea that a handful of informative tagging SNPs could capture most of the haplotypic diversity in a haplotype block of reasonable size or length. We suggest that genotyping 5 tagging SNPs in the gene region is enough to cover more than 95% of chromosomes, without loss of power. These five tagging SNPs are rs689466, rs12042763, rs2725559, rs10911902 (or rs5275) and rs6681231 (rs20417). In particular, marker rs689466 is one of the unique SNPs, which does not tag any other known SNP markers; neither can it be tagged by any other markers even in the extended 500 kb region. Marker rs12042763 can only be tagged by a marker at ˜95 kb downstream; marker rs2725559 is able to tag a marker at as near as <3 kb and as far as 195 kb downstream; and markers rs10911902 and rs6681231 can be tagged by several markers both upstream and downstream.

The patterns and frequency distributions of the extended haplotypes may display differently in cancer patients, that is a patient possessing a particular haplotype might present a different onset of the disease or could respond differently to cancer therapy. In total, 18 SNPs (both the 12 selected tagging and 6 SNPs from the literature) have been genotyped in the first 706 colorectal cancer patients treated with placebo or rofecoxib in the VICTOR trial. The preliminary analyses show that a patient carrying haplotype 1 is 2.0 fold more likely relapsed above 65 years old than other relapsed patients, about 70% chance of recurrence, with OR1.7 (95% CI 0.75-3.84). In contrast, a patient carrying haplotype 2 is 5 fold more likely relapsed below 65 years old than other relapsed patients carry other type of haplotypes, about 80% chance of recurrence, with OR 4.5 (95% CI 1.65-12.42).

### Example 2

Approximately half of all patients undergoing potentially curative surgery for colorectal cancer ultimately relapse and die of metastatic disease. This has led to the introduction of adjuvant chemotherapy, the benefits of which are stage dependent and relatively small (4 to 10% improvement in 5-year survival). Cyclo-oxygenase-2 (COX-2) plays an important role in colorectal carcinogenesis during the transition from adenoma to carcinoma and in invasion, angiogenesis and metastasis. Immunocytochemical analysis of CRC tissue suggests that 70% of tumours express COX-2; which increases with stage progression and correlates with co-expression of matrix metalloproteinase-2 (MMP) and vascular endothelial growth factor. It has been demonstrated that NSAID induced reduction in polyp recurrence was greater in adenomas with high levels of COX-2 expression. Similarly, it has been found that aspirin use significantly reduced CRC risk in tumours overexpressing COX-2 (RR 0.64: 0.52-0.78), but not in cancers with weak or absent COX-2 expression (RR 0.96: 0.73-1.26). We hypothesise that rofecoxib (R) would provide a safe approach to blocking the COX-2 pathway and reduce the rate of tumour recurrence in patients who had undergone potentially curative surgery for CRC. The VICTOR trial was stopped in September 2004 as R was withdrawn due to concerns about its cardiovascular toxicity. The following presents efficacy analyses with comparisons of overall survival (OS) and disease-free survival (DFS) for R versus placebo (P).

### Methods

This was a phase III randomised, placebo-controlled double-blind trial of R in patients after potentially-curative surgery and completion of adjuvant therapy for stage II/III CRC. 7000 patients were planned to receive 25 mg R daily or an identical placebo (P) for 2 or 5 years, however the trial was terminated early after the worldwide withdrawal of R. A revised protocol and statistical analysis plan permitted detection of a reduction (HR=0.75) in risk of death with 87% power, with one pre-planned event-driven interim analysis.

### Patients

Patients were randomly assigned to receive R or P at 151 hospitals in the United Kingdom. Inclusion criteria included: histologically proven stage II and III colorectal carcinoma in patients who had undergone complete resection of the primary tumour; WHO Performance Status 0 or 1; haematological, liver and renal function within the normal range. All patients had completed potentially curative therapy (surgery+/−radiotherapy+/−chemotherapy) 12 or fewer weeks previously and had given written informed consent. Patients with active peptic ulceration or gastrointestinal bleeding in the past year, a history of adverse reactions to NSAIDs, or a known sensitivity to R were excluded, as were those receiving long-term NSAID therapy (except for low-dose aspirin, <100 mg per day), those younger than 18 years, and women who were pregnant, lactating, or premenopausal but not using contraception. Patients with a history of cancer (other than adequately treated in situ carcinoma of the cervix, basal or squamous-cell carcinoma), inflammatory bowel disease, or severe congestive heart failure were also excluded. Patients who had stable angina or who had a myocardial infarct (MI) or transient ischaemic attack (TIA) more than 6 months earlier were eligible.

### Trial Design

It was planned to randomly allocate R (one 25-mg tablet daily) or identical P to 7000 patients, with half of each group receiving the study drug for 2 or 5 years. Suitable patients were randomly assigned in a double blind fashion to R or P through the VICTOR Trial Office, which supplied drugs to participating hospitals every 6 months. Minimisation was used to balance allocations by stage (II, III), disease site (colon, rectum, recto-sigmoid), age (<50, 50-59, 60-69, 70+) prior adjuvant chemotherapy and radiotherapy.

### Protocol Modifications

Data-collection forms were amended to solicit baseline data on cardiovascular risk factors, for all patients After worldwide withdrawal of rofecoxib all investigators and patients were informed, study treatment was stopped, and follow-up continued, at 3, 6, 12, 18 and 24 months after randomisation and annually thereafter. Patients received a colonoscopy 1-2 years after primary surgery and 2- to 3-yearly thereafter; a CT scan about 1 year after surgery; clinical examination and routine blood tests at outpatient visits. All recurrences were confirmed by CT or MRI scan and patients were flagged for survival with the UK's Office of National Statistics. Adverse event data were recorded systematically throughout the study and were reviewed by the trials team (RM, DJK and MJL) to clarify diagnoses.

### COX-2 Genotyping Methods

Genomic DNA was extracted from blood from 939 patients using QIAamp DNA extraction kits (Qiagen, Crawley, UK). All genotyping was performed using Amplifluor™ fluorescent allele specific PCR by KBiosciences, Cambridge, UK. The DNA samples were typed for the 16 SNPs identified in Example 1 (see Table 7).

### Statistical Considerations

The original study was powered to detect a reduction in risk of death between treatments for stage II and stage III separately, (hazard ratio 0.80), with type I error 0.05. The modified statistical plan, after premature closure with 2434 patients, permitted detection of a reduction in risk of death of HR 0.75, 87% power, with type I error 0.05, and one interim analysis specified according to an O'Brien-Fleming alpha-spending rule when there were 350 events. The threshold for statistical significance for OS at this analysis is p<0.01325. OS, the primary endpoint, was measured from randomisation to death from any cause. Secondary endpoints include DFS, measured from randomisation to recurrence or death from any cause and recurrence-free survival, measured from randomisation to recurrence and/or colorectal cancer death. This is the report of the interim analysis, with 368 deaths. Kaplan-Meier curves and log-rank analysis were used for comparisons of OS and DFS. Subgroups were examined using HR plots. Hazard ratios and their confidence intervals were calculated from log-rank statistics and variances. Cox's proportional hazards models were used to estimate the treatment effect adjusted by prognostic factors. All reported p-values are two-sided. For analysis of OS, patients not reported as dead were censored at their last known date alive. For analysis of DFS, patients who were alive and recurrence-free were censored at their last known recurrence-free date. For analysis of DFS in first year after randomisation, patients who were alive and recurrence-free at one year were censored at that time. Numbers of SAEs were compared using a normal approximation to the Poisson distribution.

### Results

2434 patients were recruited at 151 hospitals in the UK. One patient in the R group was found to be ineligible due to an incomplete resection and one patient in the P group was ineligible having been found to have recurrent disease before randomisation. One patient in each group was ineligible because randomisation was more than 12 weeks after surgery. The cancer site of one patient randomised to P was subsequently found to be ileum. These patients were included in the intention-to-treat population used for outcome analysis, comprising 1217 patients randomized to R and 1217 to P. One patient randomized to P, and another to R received the incorrect treatment for the first 6 months and one randomized to R switched over to P for a period of 3 weeks before switching back to R. 50 patients randomized to R and 57 randomised to P had not yet started treatment when the drug was withdrawn. The treated population therefore comprised 1167 R and 1160 P patients). Assignment of study treatment was balanced on the basis of gender, disease site, stage, age and prior adjuvant chemotherapy and radiotherapy (table 15). Slightly more patients in the R group were using low-dose aspirin at the time of randomisation (8.6% v 6.9%). The median time on treatment was 7.4 months (inter-quartile range 3.1 to 14.0), and 8.2 months (inter-quartile range 3.7 to 14.9) respectively for R and P, with 33% having completed at least 12 months. 18 patients randomised to 2 years R and 23 randomised to 2 years P were reported as having completed their study treatment. There were 806 patients still on R and 862 still on P when the drug was withdrawn. The difference in the duration of treatment was related to a trend towards a higher proportion of early discontinuations in the R arm for side effects. The most common medical reasons for early discontinuation of study drug were gastrointestinal pain or heartburn (15 R and 5 P), analgesia required for arthritis (4 R and 15 P), hypertension (7 R and 1 P), renal impairment (7 R and 1 P), diarrhea (4 R and 4 P) and heart failure (2R).

### Safety (Treated Population)

Serious Adverse Events (SAEs) were collected for each patient from enrolment until the data lock of November 2007. Cardiovascular thrombotic events have previously been reported in detail. In summary, of 23 confirmed cardiovascular thrombotic events that occurred within the treatment period or within 14 days after cessation, 16 occurred in the R arm and 7 in the P arm with an estimated relative risk of 2.66 (95% confidence intervals (CI) 1.03-6.86; p=0.04). These included fatal and nonfatal myocardial infarction, unstable angina, sudden death from cardiac causes, fatal and nonfatal ischemic stroke, transient ischemic attack, peripheral arterial thrombosis, peripheral venous thrombosis, and pulmonary embolism. Fourteen more cardiovascular thrombotic events, six in the R arm and eight in the P arm, were reported within the two years after the trial closure, with an overall total period unadjusted relative risk of 1.50 (95% CI 0.76-2.94; p=0.24). Other cardiovascular SAEs reported included fatal haemorrhagic stroke (1 P), ruptured cerebral aneurysm (1 P), cardiac arrhythmias (4 P and 3 R), stable angina (2 P and 5 R), cardiac failure of unknown aetiology (1 P and 4 R), and hypertension (2R); none of these differed significantly in frequency between the 2 arms of the trial. Gastro-intestinal SAEs included indigestion/gastroduodenal ulceration, diarrhoea, constipation and obstruction due to probable adhesions, all of which were reported in less than 1% of patients with no significant difference in frequency comparing R and P. Various infections were reported with a slight preponderance in the R arm (10 R and 3 P; p=0.05) but with no consistent pattern of site or organism. Other SAEs reported included depression, neuralgia, polymyositis and epilepsy, at a frequency of less than 0.2%. Taking into account all-cause SAEs, there was an association with R administration (p=0.05).

### Overall Survival

Median follow up was 36.4 (inter-quartile range 29.5-45.4) months for the R group and 36.7 (inter-quartile range 28.3-46.6) months for the P group. Over the study period there were 177 deaths in the R group and 191 in the P group. The numbers of deaths without recurrent disease were 15 and 14 in the R and P groups respectively. Five patient deaths in each group had no reported cause. The HR for dying from any cause with R compared with P was 0.94 (95% CI, 0.77 to 1.16, P=0.57). The HR for colorectal cancer-specific mortality was 0.93 (95% CI 0.75-1.15; p=0.50). The 3-year Kaplan-Meier OS rates were 87.6 (95% CI, 85.7 to 89.6) for the R group and 86.6 (95% CI, 84.5 to 88.6) for the P group, FIG. 5. Survival data is also shown in the table below. The effect of P on OS in stage, site, age, gender, and adjuvant chemotherapy subgroups shows little evidence of variation. A proportional hazards analysis showed that the HR for dying with R when adjusted for stage, age, radiotherapy group and aspirin use at baseline was little changed from the unadjusted figure at 0.95 (95% CI 0.78-1.17; p=0.64).

Tabulated Data corresponding to FIG. 5 indicating number of patients still at risk for each time point:

Years from randomisation

| | 0 | 1 | 2 | 3 | 4 | 5 |
|---|---|---|---|---|---|---|
| Rofecoxib | 1217 | 1152 | 1012 | 663 | 219 | 21 |
| Placebo | 1217 | 1153 | 1005 | 684 | 236 | 30 |

### Disease Free Survival

There were 291 DFS events in the R group and 316 in the P group, FIG. 6. Disease free survival data is also shown in the table below. The HR for disease recurrence or death from any cause with R compared with P was 0.91 (95% CI, 0.78 to 1.07, P=0.25). The HR for colorectal cancer recurrence was 0.90 (95% CI 0.77-1.06; p=0.22) with 271 and 297 events in the R and P arms respectively. In absolute terms, the 3-year DFS rates were 74.7% (95% CI 72.0 to 77.3) for the R group and 72.6% (95% CI 69.9 to 75.3) for the P group. Adjustment for stage, age, radiotherapy group and gender did not change the HR for death or recurrence with R versus P remaining at 0.91 (95% CI, 0.78, 1.07, p=0.30). In a non-prespecified analysis, there is a difference in DFS comparing the R group and the P group in the first year at risk from randomisation with a trend towards significance (117 vs 145 events, p=0.07) which is lost on subsequent follow up (year 2 onwards, 174 vs 171 events, p=0.97).

Tabulated Data corresponding to FIG. 6 indicating number of patients still at risk for each time point:

Years from randomisation

| | 0 | 1 | 2 | 3 | 4 | 5 |
|---|---|---|---|---|---|---|
| Rofecoxib | 1217 | 1031 | 824 | 449 | 114 | 7 |
| Placebo | 1216 | 1014 | 799 | 455 | 133 | 12 |

### COX-2 Genotype

3 SNPs (rs 10911907 [SNP ID no. 17], rs11583191 [SNP ID no. 14] and rs2179555 [SNP ID no. 16]) which all lie 5′ to the PTGS2 gene and are in high linkage disequilibrium with each other (D′>0.9, pair wise comparisons), were identified in a COX multivariate model as conferring a poor prognosis HR 1.57 (0.99-2.48, p=0.06). Out of 870 patients genotyped for these three SNPs, 649 have wild type alleles at each locus and 214 (24.6%) carry at least one variant allele at each locus. The effect of R on delaying recurrence is significantly greater in those patients who have three variant alleles (p-value for treatment interaction=0.04) after adjustment for known prognostic factors (stage of disease, radiotherapy, age). The unadjusted recurrence free survival curves stratified by treatment and compound genotype are shown in FIG. 7. Survival data is also shown in the Table below.

Tabulated Data corresponding to FIG. 7 indicating number of patients still at risk for each time point:

| | Years from randomisation | | | | | |
|---|---|---|---|---|---|---|
| | 0 | 1 | 2 | 3 | 4 | 5 |
| Variant, rofecoxib | 105 | 98 | 92 | 53 | 12 | 1 |
| Variant, placebo | 109 | 99 | 82 | 51 | 16 | 3 |
| Wild-type, rofecoxib | 347 | 330 | 281 | 172 | 44 | 5 |
| Wild-type, placebo | 302 | 284 | 230 | 138 | 41 | 4 |

### Summary of Results

1167 patients received R and 1160 received P for median treatment durations of 7.4 months and 8.2 months respectively. Median follow-up was 3.0 and 3.1 years (R vs P), with 177 vs 191 deaths and 291 vs 316 recurrences. The pre-planned analyses demonstrated no difference in overall survival (OS), HR 0.94 (95% CI 0.77-1.16; p=0.57), or DFS, HR 0.91 (95% CI 0.78-1.07; p=0.25), comparing the two groups. 870 patients were genotyped using the SNPs from Example 1. 3 COX-2 SNPs were associated with poor prognosis and a positive treatment interaction (p=0.04) in favour of R. Thus, the presence of the minor alleles of SNP ID nos. 14, 16 and 17 in the genotype of an individual is associated with poor prognosis and a positive treatment interaction with a COX-2 inhibitor (R).

### Discussion

It is reasoned that blockade of the biochemical pathway activated by COX-2 would induce the following downstream events; direct inhibition of growth of residual micrometastases which had spread prior to surgery; prevention of angiogenesis; reduction in tumoural expression of the matrix metalloproteinases required for further tissue invasion. Given the relative gastrointestinal safety of R, we initially estimated that a small (3%) absolute improvement in 5-year survival, if proven, would be sufficient to warrant use of this agent in the adjuvant setting.

Results from the specified analyses show no overall benefit for R, with no obvious improvements in disease-free or overall survival. Although the trial recruited 2434 patients, there are two potential reasons why a statistically significant benefit was not established; there were insufficient patient numbers to demonstrate a clinically relevant but small effect; the duration of effective COX-2 inhibition may have been inadequate to alter the malignant phenotype. There are data from the aspirin colorectal cancer prevention studies to suggest a dose and duration effect with respect to cancer risk reduction and adenoma prevention studies with R and celecoxib exposed patients to 16-36 months drug treatment. The premature closure of this trial and abbreviated duration of treatment may have attenuated therapeutic benefit.

The greatest number of colorectal cancer recurrences occur in the first year following surgery and the majority of adjuvant chemotherapy benefits are accrued then, with little demonstrable therapeutic effect in subsequent years. In this study, more recurrences were seen in the P arm than in the R group (141 vs 112, p=0.06) in the first year, with a slight rebound in year 2 (88 vs 99 recurrences). Median drug exposure was 7.4 months, which allows the speculation that upon premature R withdrawal, any inhibitory effects on tumoural production of proteins like VEGF and MMP would cease, releasing the “cytostatic brake” from residual micrometastatic deposits, prompting a higher rate of progression and recurrence in the R arm in following years.

COX-2 SNPs were genotyped in 870 patients and showed that three SNPs identified a subgroup of patients with a poor prognosis (HR 1.57, p=0.06) for whom the R response was significantly superior (p=0.04) to P (FIG. 7). Many investigators have found a strong correlation between COX-2 expression and prognosis, in a range of other solid tumours including prostate and lung cancer, and the pathway remains a continuing focus of clinical and translational research.

In general, R was well tolerated apart from the enhanced cardiovascular adverse event profile (1-2%), which needs to be considered in the context of the cardiotoxicity (2-4%) that is observed with conventional cytotoxics such as 5-fluorouracil in the adjuvant setting. If further trials are to be performed exploring the role of COX-2 inhibition in cancer patients, attention must be given to maintaining the duration of therapy, considering carefully what size of clinical benefit would be recognised as worthwhile. The SNPs identified in this study may be used to select a patient population who would benefit most from treatment with a COX-2 inhibitor.

### CONCLUSIONS

In this study of truncated treatment duration, therapy with R is unlikely to have resulted in a substantial improvement in OS or in protection from recurrence of CRC. However, COX-2 genotyping identifies a subgroup of responsive patients, who would benefit most from treatment with a COX-2 inhibitor.

TABLE 15
Baseline characteristics of randomised patients

| | Rofecoxib | Placebo |
|---|---|---|
| | (N = 1217) | (N = 1217) |
| | N(%) | N(%) |
| Minimisation variables | | |
| Colon | 791 (65.0) | 802 (65.9) |
| Rectum | 329 (27.0) | 332 (27.3) |
| Junction | 97 (8.0) | 83 (6.8) |
| Dukes B | 579 (47.6) | 580 (47.7) |
| Dukes C | 638 (52.4) | 637 (52.3) |
| Age <50 | 294 (24.2) | 296 (24.3) |
| Age 50-59 | 470 (38.6) | 470 (38.6) |
| Age 60-69 | 362 (29.7) | 361 (29.7) |
| Age 70+ | 91 (7.5) | 90 (7.4) |
| Bolus 5FU | 671 (55.1) | 677 (55.6) |
| Infusional 5FU or oral | 109 (9.0) | 107 (8.8) |
| New agent with or without 5FU | 8 (0.7) | 7 (0.6) |
| No chemotherapy | 429 (35.3) | 426 (35.0) |
| Other entry characteristics | | |
| Males | 782 (64.3) | 779 (64.0) |
| Females | 435 (35.7) | 438 (36.0) |
| Ethnic: White | 1193 (98.0) | 1193 (98.0) |
| Ethnic group: Other | 14 (1.2) | 16 (1.3) |
| Ethnic group: NK | 10 (0.8) | 8 (0.7) |
| Preoperative Radiotherapy | 122 (10.0) | 132 (10.8) |
| Postoperative Radiotherapy | 21 (1.7) | 24 (2.0) |
| No Radiotherapy | 1074 (88.2) | 1061 (87.2) |
| Long-term low-dose aspirin | 105 (8.6) | 84 (6.9) |
| use at randomisation | | |

1. A method for determining whether an individual has, or is susceptible to a disease selected from a cancer, a cardiovascular disease or a disease that involves the immune system comprising determining whether any of haplotypes 1 to 10 as shown in Table 7 are present in or absent from the genome of the individual.

2. A method according to claim 1 comprising:
determining whether 3 or more, or all of the polymorphisms in any single row of Table 7 are present in or absent from the genome of the individual, and/or
determining whether 1, 2, 3, 4 or all of haplotypes 1 to 10 are present in or absent from the genome of the individual, and/or
typing 3 or more, or all of the nucleotide positions at which the polymorphisms in Table 7 occur, and/or
typing both of the chromosomes of the individual at any of the nucleotide positions at which the polymorphisms in Table 7 occur.

3. A method according to claim 1, wherein the disease is recurrence of a cancer, osteoarthritis, Alzheimers disease, an infectious disease, a cardiovascular disease, an inflammatory disease, a musculoskeletal disease or a disease that involves the immune system.

4. A method according to claim 1 wherein the cancer is colorectal cancer, breast cancer, prostate cancer, esophageal cancer, stomach cancer, liver cancer or lung cancer.

5. A method according to claim 1 wherein both of the chromosomes are typed, wherein the absence or presence of:
i) haplotype 1 and haplotype 4; or
ii) haplotype 1 and haplotype 5; or
iii) haplotype 1 and haplotype 9; or
iv) haplotype 2 and haplotype 6;
is determined to ascertain whether the subject is more likely to progress to stage III colorectal cancer.

6. A method according to claim 1 wherein both of the chromosomes are typed and the absence or presence of:
i) haplotype 1 and haplotype 5; or
ii) haplotype 3 and haplotype 5; or
iii) haplotype 5 and haplotype 7; or
iv) haplotype 5 and haplotype 8; or
v) haplotype 5 and haplotype 9; or
vi) haplotype 5 and haplotype 10
is determined to ascertain whether the subject is more likely to progress to stage III colorectal cancer, wherein optionally the individual is over 65 years of age.

7. A method according to claim 1 comprising determining whether the individual possesses:
(i) haplotype 1,
(ii) haplotype 2,
and thereby determining whether the individual is at risk of recurrence of cancer (relapse).

8. A method according to claim 7 wherein both of the chromosomes are typed and the absence or presence of:
(i) haplotype 1 and any one of haplotypes 3 to 10; and/or
(ii) haplotype 2 and any one of haplotypes 3 to 10
is determined.

9. A method according to claim 7(i) wherein the individual is over 65 years old or a method according to claim 7(ii) wherein the individual is less than 65 years old.

10. A non-human cell comprising a polynucleotide that comprises any of haplotypes 1 to 10 as shown in Table 7.

11. A kit for carrying out the method of claim 1 comprising polynucleotides for detecting one or more of haplotypes 1 to 10 as shown in Table 7.

12. A kit according to claim 7 comprising at least one polynucleotide probe or primer which is capable of detecting at least one polymorphism as defined in Table 7.

13. A polynucleotide array for determining the extended PTGS2 haplotype of a subject, comprising a means for detecting one or more of the haplotypes shown in Table 7, wherein the array optionally comprises at least one polynucleotide probe or primer which is capable of detecting at least one polymorphism as defined in Table 7.

14. A method of screening for a therapeutic substance which treats a disease as mentioned in claim 1 comprising contacting a candidate substance with a vector that comprises the sequence of any of haplotypes 1 to 10 and determining whether the candidate substance is able to modulate expression from the vector.

15. A therapeutic substance which prevents or treats a disease as defined in claim 1 for use in a method of treatment comprising determining whether an individual has or is susceptible to a disease by a method according to claim 1, and if the individual is determined to have the disease or be susceptible comprising administering the therapeutic substance to the individual.

16. A method of typing an informative extended PTGS2 haplotype of individual comprising determining the absence of presence of any of haplotypes 1 to 10 in an individual.

17. A method for determining:
whether an individual has, or is susceptible to colorectal cancer; and/or
whether an individual will respond positively to COX-2 inhibitor treatment, comprising determining whether the minor alleles of each of the SNPs 14, 16 and 17 (rs11583191, rs2179555 and rs10911907) are present in or absent from the genome of the individual.

18. A method for treating colorectal cancer in an individual, comprising:
determining whether the minor alleles of each of the SNPs 14, 16 and 17 (rs11583191, rs2179555 and rs10911907) are present in or absent from the genome of the individual; and
when each of the three minor alleles is present, administering a COX-2 inhibitor to the individual.

19. (canceled)

20. The method of claim 17, wherein the COX-2 inhibitor is rofecoxib.

21. The method of claim 18, wherein the COX-2 Inhibitor is rofecoxib.


SEQUENCE LISTING (73 sequences)

SEQ ID NO: 1 - DNA, 4465 bp, Homo sapiens
caattgtcatacgacttgcagtgagcgtcaggagcacgtccaggaactcctcagcagcgcctccttcagctccacagccagacgccctcagacagcaaagcctacccccgcgccgcgccctgcccgccgctcggatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtcttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactatcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagatgaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatctcaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataacgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatattttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactattta

SEQ ID NO: 2 - DNA, 140 bp, Homo sapiens
ccccaacacacaaaaacaattataacaaaaataaacatattctggctcttaatgggtggtcctatgtttgtgatcagtgtyagtgattagtgcagattctaggactattttttttttcctttcataacatgcagtgaaaa

SEQ ID NO: 3 - DNA, 139 bp, Homo sapiens
cttttgggaagagggagaaaatgaaataaatatcattaaagataactcaggagaatcttctttacaattttacrtttagaatgtttaaggttaagaaagaaatagtcaatatgcttgtataaaacactgttcactgttt

SEQ ID NO: 4 - DNA, 140 bp, Homo sapiens
ctgacatttaatggtactgtatattacttaatttattgaagattattatttatgtcttattaggacactatggttataarctgtgtttaagcctacaatcattgatttttttttgttatgtcacaatcagtatattttct

SEQ ID NO: 5 - DNA, 140 bp, Homo sapiens
ttatgaggtcattgctacttttgcaatgtgatatggactgctaaattaaactgtacaacagaaaatctgagaaaacatatcrttattcaagcacagcttggtacttcattaacctcatagcaaaatctgagtaccaggcc

SEQ ID NO: 6 - DNA, 140 bp, Homo sapiens
ttacactgtcgatgtttccaatgcatcttccatgatgcattagaagtaactaatgtttgaaattttaaagtacttttggtyatttttctgtcatcaaacaaaarcaggtatcagtgcattattaaatgaatatttaaatt

SEQ ID NO: 7 - DNA, 137 bp, Homo sapiens
tcttcatcgccttcacaggagaaaaggaaatgtctgcagagttggaagcactctatggtgacatcgatgctgtggrgctgtatcctgcccttctggtagaaaagcctcggccagatgccatctttggtgaaaccatg

SEQ ID NO: 8 - DNA, 140 bp, Homo sapiens
tattttttggatttcaatagcatagcttcaagttattcgtaagaattttttataaataatacatttttatacttttttakaattaccatatcatcatagtgaagtatataatatatatgatataagctcaatatagtata

SEQ ID NO: 9 - DNA, 140 bp, Homo sapiens
acatacttacccacttcaagggattttggaacgttgtgaataacattcccttccttcgaaatgcaattatgagttatgtsttgacatgtaagtacaagtgtctttctaaggtttttagccttctcaaagaaaaatatgct

SEQ ID NO: 10 - DNA, 140 bp, Homo sapiens
caggatctgatcaatatatgtgaattgtttatatttggaacctttttattgagtggaagaagttgttttaaatattctartcagttctttcctgctcccaggaaagcccggattatgttttaagataagcaaaatgtctt

SEQ ID NO: 11 - DNA, 140 bp, Homo sapiens
tggtgacccgtggagctcacattaactatttacagggtaactgcttaggaccagtattatgaggagaatttacctttcccscctctctttccaagaaacaaggagggggtgaaggtacggagaacagtatttcttctgtt

SEQ ID NO: 12 - DNA, 140 bp, Homo sapiens
tttagtatctcaccctcacatgctcctccctgagcactacccatgatagatgttaaacaaaagcaaagatgaaattccarctgtcaaaatctcccttccatctaattaattcctcatccaactatgttccaaaacgagaa

SEQ ID NO: 13 - DNA, 140 bp, Homo sapiens
aatgtgccaaactaaattaagaccactaaacctgttttatatggaaaagtactgtgactattgtgtaccataaaaaaaagktttgatcatgtaaaacttatagctctcatgtttgcatatattcaatgtttctgtgtcta

SEQ ID NO: 14 - DNA, 140 bp, Homo sapiens
tgtttctgtgtctattttaggcaaatatatggatctgttgtacaaaatttctattagcgtctgaaatttgttgggaaatamtctacatgaaaagaaaataaggaagaatcttaaaaatcttccagagctttctgttgagt

SEQ ID NO: 15 - DNA, 140 bp, Homo sapiens
ctgtcgggatttttgtcgcagcctaactgaattagaagaaggaaaatatccaaatttaactgcctctagctttccatacargggaagaaaaatacccaactccagccctctctttagcttctacattggagaagggaaat

SEQ ID NO: 16 - DNA, 140 bp, Homo sapiens
gaaagtcaccgttcataggcacaggctcactaaaaggccgagacctgctataggactatagaatgcttcacttcccctgamctcttatcgtcacattacaatgtattgagtttctttcactcagtacatcatgcctggct

SEQ ID NO: 17 - DNA, 140 bp, Homo sapiens
gtaagtagacagatggcaattccaagaaataatcagagaaatgctagagatcaaaaacactggaacagaagtcaacaatgyctgtaagggcttattagtagactggacatggcagaggaaagaatccctgcgattgcaga

SEQ ID NO: 18 - DNA, 140 bp, Homo sapiens
tcaatcaaatcaaaaacagaaaatcaaaaaagaaaatgggtaaaaccaaaaaggtggttcttttaaaagacataaagttrattactctagtcaggctaaggaaaaaaagagaagacacaaattattagtatcagaattga

SEQ ID NO: 19 - DNA, 140 bp, Homo sapiens
tcaaaaacttggaaacaatcaagatgtacttcagaaggagaacggattaattgtgatatattcagtcagtagagcattastcagcagtaaaagaaaatgtggtaccatcatatatttaagacatggaggaatcttaaata

SEQ ID NO: 20 - DNA, 47 bp, Artificial
  rs10911902 primer (allele 1)
gaaggtgaccaagttcatgctagtcctagaatctgcactaatcacta

SEQ ID NO: 21 - DNA, 46 bp, Artificial
  rs10911902 primer (allele 2)
gaaggtcggagtcaacggattgtcctagaatctgcactaatcactg

SEQ ID NO: 22 - DNA, 28 bp, Artificial
  rs10911902 primer (common)
tcttaatgggtggtcctatgtttgtgat

SEQ ID NO: 23 - DNA, 54 bp, Artificial
  rs689469 primer (allele 1)
gaaggtgaccaagttcatgctactatttctttcttaaccttaaacattctaaat

SEQ ID NO: 24 - DNA, 54 bp, Artificial
  rs689469 primer (allele 2)
gaaggtcggagtcaacggattactatttctttcttaaccttaaacattctaaac

SEQ ID NO: 25 - DNA, 30 bp, Artificial
  rs689469 primer (common)
cttttgggaagagggagaaaatgaaataaa

SEQ ID NO: 26 - DNA, 51 bp, Artificial
  rs4648298 primer (allele 1)
gaaggtgaccaagttcatgctaaaatcaatgattgtaggcttaaacacagt

SEQ ID NO: 27 - DNA, 48 bp, Artificial
  rs4648298 primer (allele 2)
gaaggtcggagtcaacggattatcaatgattgtaggcttaaacacagc

SEQ ID NO: 28 - DNA, 30 bp, Artificial
  rs4648298 primer (common)
ttatgtcttattaggacactatggttataa

SEQ ID NO: 29 - DNA, 51 bp, Artificial
  rs2206593 primer (allele 1)
gaaggtgaccaagttcatgctgtacaacagaaaatctgagaaaacatatca

SEQ ID NO: 30 - DNA, 49 bp, Artificial
  rs2206593 primer (allele 2)
gaaggtcggagtcaacggattacaacagaaaatctgagaaaacatatcg

SEQ ID NO: 31 - DNA, 30 bp, Artificial
  rs2206593 primer (common)
gattttgctatgaggttaatgaagtaccaa

SEQ ID NO: 32 - DNA, 54 bp, Artificial
  rs5275 primer (allele 1)
gaaggtgaccaagttcatgctactaatgtttgaaattttaaagtacttttggtt

SEQ ID NO: 33 - DNA, 54 bp, Artificial
  rs5275 primer (allele 2)
gaaggtcggagtcaacggattactaatgtttgaaattttaaagtacttttggtc

SEQ ID NO: 34 - DNA, 30 bp, Artificial
  rs5275 primer (common)
ttaataatgcactgatacctgyttttgttt

SEQ ID NO: 35 - DNA, 43 bp, Artificial
  rs5272 primer (allele 1)
gaaggtgaccaagttcatgctatggtgacatcgatgctgtgga

SEQ ID NO: 36 - DNA, 43 bp, Artificial
  rs5272 primer (allele 2)
gaaggtcggagtcaacggattatggtgacatcgatgctgtggg

SEQ ID NO: 37 - DNA, 25 bp, Artificial
  rs5272 primer (common)
gcttttctaccagaagggcaggata

SEQ ID NO: 38 - DNA, 54 bp, Artificial
  rs20432 primer (allele 1)
gaaggtgaccaagttcatgctattatatacttcactatgatgatatggtaattc

SEQ ID NO: 39 - DNA, 51 bp, Artificial
  rs20432 primer (allele 2)
gaaggtcggagtcaacggattatatacttcactatgatgatatggtaatta

SEQ ID NO: 40 - DNA, 30 bp, Artificial
  rs20432 primer (common)
tggatttcaatagcatagcttcaagttatt

SEQ ID NO: 41 - DNA, 48 bp, Artificial
  rs5277 primer (allele 1)
gaaggtgaccaagttcatgctgaaagacacttgtacttacatgtcaac

SEQ ID NO: 42 - DNA, 48 bp, Artificial
  rs5277 primer (allele 2)
gaaggtcggagtcaacggattgaaagacacttgtacttacatgtcaag

SEQ ID NO: 43 - DNA, 29 bp, Artificial
  rs5277 primer (common)
cccttccttcgaaatgcaattatgagtta

SEQ ID NO: 44 - DNA, 54 bp, Artificial
  rs4648261 primer (allele 1)
gaaggtgaccaagttcatgctattgagtggaagaagttgttttaaatattctaa

SEQ ID NO: 45 - DNA, 51 bp, Artificial
  rs4648261 primer (allele 2)
gaaggtcggagtcaacggattgagtggaagaagttgttttaaatattctag

SEQ ID NO: 46 - DNA, 30 bp, Artificial
  rs4648261 primer (common)
ttgcttatcttaaaacataatccgggcttt

SEQ ID NO: 47 - DNA, 44 bp, Artificial
  rs20417 primer (allele 1)
gaaggtgaccaagttcatgctccttgtttcttggaaagagaggc

SEQ ID NO: 48 - DNA, 44 bp, Artificial
  rs20417 primer (allele 2)
gaaggtcggagtcaacggattccttgtttcttggaaagagaggg

SEQ ID NO: 49 - DNA, 29 bp, Artificial
  rs20417 primer (common)
ggaccagtattatgaggagaatttacctt

SEQ ID NO: 50 - DNA, 48 bp, Artificial
  rs689466 primer (allele 1)
gaaggtgaccaagttcatgctattagatggaagggagattttgacagt

SEQ ID NO: 51 - DNA, 45 bp, Artificial
  rs689466 primer (allele 2)
gaaggtcggagtcaacggattagatggaagggagattttgacagc

SEQ ID NO: 52 - DNA, 29 bp, Artificial
  rs689466 primer (common)
gagcactacccatgatagatgttaaacaa

SEQ ID NO: 53 - DNA, 51 bp, Artificial
  rs12042763 primer (allele 1)
gaaggtgaccaagttcatgctatgagagctataagttttacatgatcaaac

SEQ ID NO: 54 - DNA, 52 bp, Artificial
  rs12042763 primer (allele 2)
gaaggtcggagtcaacggattcatgagagctataagttttacatgatcaaaa

SEQ ID NO: 55 - DNA, 29 bp, Artificial
  rs12042763 primer (common)
ggaaaagtactgtgactattgtgtaccat

SEQ ID NO: 56 - DNA, 53 bp, Artificial
  rs2745559 primer (allele 1)
gaaggtgaccaagttcatgctaagattcttccttattttcttttcatgtagat

SEQ ID NO: 57 - DNA, 47 bp, Artificial
  rs2745559 primer (allele 2)
gaaggtcggagtcaacggattcttccttattttcttttcatgtagag

SEQ ID NO: 58 - DNA, 30 bp, Artificial
  rs2745559 primer (common)
gttgtacaaaatttctattagcgtctgaaa

SEQ ID NO: 59 - DNA, 47 bp, Artificial
  rs11583191 primer (alelle 1)
gaaggtgaccaagttcatgctttaactgcctctagctttccatacaa

SEQ ID NO: 60 - DNA, 45 bp, Artificial
  rs11583191 primer (allele 2)
gaaggtcggagtcaacggattaactgcctctagctttccatacag

SEQ ID NO: 61 - DNA, 26 bp, Artificial
  rs11583191 primer (common)
gagggctggagttgggtatttttctt

SEQ ID NO: 62 - DNA, 49 bp, Artificial
  rs2143416 primer (allele 1)
gaaggtgaccaagttcatgctcaatacattgtaatgtgacgataagagt

SEQ ID NO: 63 - DNA, 49 bp, Artificial
  rs2143416 primer (allele 2)
gaaggtcggagtcaacggattcaatacattgtaatgtgacgataagagg

SEQ ID NO: 64 - DNA, 29 bp, Artificial
  rs2143416 primer (common)
ctgctataggactatagaatgcttcactt

SEQ ID NO: 65 - DNA, 48 bp, Artificial
  rs2179555 primer (allele 1)
gaaggtgaccaagttcatgctaaacactggaacagaagtcaacaatgt

SEQ ID NO: 66 - DNA, 46 bp, Artificial
  rs2179555 primer (allele 2)
gaaggtcggagtcaacggattacactggaacagaagtcaacaatgc

SEQ ID NO: 67 - DNA, 25 bp, Artificial
  rs2179555 primer (common)
cctctgccatgtccagtctactaat

SEQ ID NO: 68 - DNA, 54 bp, Artificial
  rs10911907 primer (allele 1)
gaaggtgaccaagttcatgctaaaaaggtggttcttttaaaagacataaagtta

SEQ ID NO: 69 - DNA, 51 bp, Artificial
  rs10911907 primer (allele 2)
gaaggtcggagtcaacggattaaggtggttcttttaaaagacataaagttg

SEQ ID NO: 70 - DNA, 29 bp, Artificial
  rs10911907 primer (common)
ctctttttttccttagcctgactagagta

SEQ ID NO: 71 - DNA, 49 bp, Artificial
  rs6681231 primer (allele 1)
gaaggtgaccaagttcatgctgtgatatattcagtcagtagagcattag

SEQ ID NO: 72 - DNA, 49 bp, Artificial
  rs6681231 primer (allele 2)
gaaggtcggagtcaacggattgtgatatattcagtcagtagagcattac

SEQ ID NO: 73 - DNA, 29 bp, Artificial
  rs6681231 primer (common)
gatggtaccacattttcttttactgctga